SEQUENCE LISTING (24 sequences)

SEQ ID NO: 1 - DNA, 23 bp, Artificial Sequence
  guide sequence
gctcaccaacggtctcctctcgg

SEQ ID NO: 2 - DNA, 22 bp, Artificial Sequence
  guide sequence
gttgccagaggagagccccctg

SEQ ID NO: 3 - DNA, 91 bp, Sus scrofa
gggcctctgggctcaccaacggtctcctctcgggggacgaagacttctcctccattgcggacatggacttctcagcccttctgagtcagat

SEQ ID NO: 4 - DNA, 90 bp, Artificial Sequence
  HDR template
gggcctctgggctcaccaacggtctcctcccgggggacgaagacttctcctccattgcggacatggacttctcagcccttctgagtcaga

SEQ ID NO: 5 - DNA, 15 bp, Artificial Sequence
  Left Talen
ctcctccattgcgga

SEQ ID NO: 6 - DNA, 15 bp, Artificial Sequence
  TALEN
cttctgagtcagatc

SEQ ID NO: 7 - DNA, 33 bp, Artificial Sequence
  TALENs
ggaagaagtatcagccatacagaaattctgggt

SEQ ID NO: 8 - DNA, 86 bp, sus scrofa
ccagatcgccaaatgcatggaagaagtatcagccattcatccctcccaggaagacagaaattctgggtcaaccacggagttgcact

SEQ ID NO: 9 - DNA, 23 bp, Artificial Sequence
  guide sequence
gggagggtccttctgtctttaag

SEQ ID NO: 10 - DNA, 89 bp, Artificial Sequence
  HDR template
ccagatcgccaaagtcacggaagaagtatcagccattcatccctcccagtgaacttacagaaattctgggtcgaccacggagttgcact

SEQ ID NO: 11 - DNA, 61 bp, sus scrofa
tagatggatgaaaccgaaattagaagtttctttgctagatatggttcagtaaaagaagtga

SEQ ID NO: 12 - DNA, 65 bp, Artificial Sequence
  HDR template
tagacggatgaaaccgaaattagaagttggatcctttgctagatatggttcagtaaaaggagtga

SEQ ID NO: 13 - DNA, 65 bp, Artificial Sequence; identical to SEQ ID NO: 12
  Founder swine as modified

SEQ ID NO: 14 - DNA, 29 bp, Artificial Sequence
  Founder swine as modified
tagatggatgaaaccgaaattagaagtga

SEQ ID NO: 15 - DNA, 39 bp, Artificial Sequence
  Founder Swine as modified
tagatggatgaaaccgatatggttcagtaaaagaagtga

SEQ ID NO: 16 - DNA, 34 bp, Artificial Sequence
  Founder Swine as modified
tagatggactagatatggttcagaaaagaagtga

SEQ ID NO: 17 - DNA, 61 bp, Artificial Sequence
  Founder Swqne as modified
tcatggaagaagtatcagccattcatccctcccaggaggacagaaattctgggtcaaccac

SEQ ID NO: 18 - DNA, 65 bp, Artificial Sequence
  Founder Swine as modifed
tcacggaagaagtatcagccattcatccctcccagtgaagcttacagaaattctgggtcagccac

SEQ ID NO: 19 - DNA, 65 bp, Artificial Sequence
  Founder Swine as modified
tcatggaagaagtatcagccattcatccctcccagtgaagcttacagaaattctgggtcaaccac

SEQ ID NO: 20 - DNA, 64 bp, Artificial Sequence
  Founder Swien as modfifed
tcatggaagaagtatcagccattcatccctccccccaggaagacagaaattctgggtcaaccac

SEQ ID NO: 21 - DNA, 65 bp, Artificial Sequence
  Founder Swine as modified
tcatggaagaagtatcagccattcatccctcccagtgaagcttacagaaattctgggtcagccac

SEQ ID NO: 22 - DNA, 61 bp, Artificial Sequence
  Founder Swine as modified
tcatggaagaagtattagccattcatccctcccaggaagacagaaattctgggtcaaccac

SEQ ID NO: 23 - DNA, 65 bp, Artificial Sequence
  Founder Swine as modified
tcacggaagaagtatcagccattcatccctcccagtgaagcttacagaaattctgggtcaaccac

SEQ ID NO: 24 - DNA, 58 bp, Artificial Sequence
  Founder Swine as modified
tcatggaagaagtatcagccattcatccctccgaagacagaaactctgggtcaaccac

(19) United States
(12) Patent Application Publication
(10) Pub. No.: US 2014/0359796 A1
(43) Pub. Date: Dec. 4, 2014

(54) GENETICALLY STERILE ANIMALS

(71) Applicant: Recombinetics, Inc., Saint Paul, MN (US)

(72) Inventors: Scott C. Fahrenkrug, Minneapolis, MN (US); Daniel F. Carlson, Woodbury, MN (US)

(21) Appl. No.: 14/263,408

(22) Filed: Apr. 28, 2014

Related U.S. Application Data
(60) Provisional application No. 61/870,558, filed on Aug. 27, 2013.
(60) Provisional application No. 61/829,656, filed on May 31, 2013.

(51) Int. Cl. A01K 67/027; C12N 15/90

(52) U.S. Cl. CPC A01K 67/0276; C12N 15/907; C12N 2800/80; A01K 2227/40; A01K 2227/30; A01K 2227/106; A01K 2227/101; A01K 2227/102; A01K 2227/103; A01K 2227/10; A01K 2227/108
USPC 800/15; 435/462; 435/325; 800/13; 800/19; 800/14; 800/16; 800/17; 800/20

(57) ABSTRACT

A genetically modified livestock animal, and methods of making and using the same, the animal comprising a genetic modification to disrupt a target gene selectively involved in gametogenesis, wherein the disruption of the target gene prevents formation of functional gametes of the animal. Animals that create progeny with donor genetics, and methods of making and using the same. Cells, and methods of making and using the cells, with a genetic modification to disrupt a target gene selectively involved in gametogenesis.

### CROSS-REFERENCE TO RELATED APPLICATIONS

This application claims priority to U.S. Ser. No. 61/870,558 filed Aug. 27, 2013 and U.S. Ser. No. 61/829,656 filed May 31, 2013, each of which are hereby incorporated by reference herein.

### STATEMENT OF GOVERNMENT SUPPORT

Aspects of the work described herein were supported by grant 1R43RR033149-01A1 from the National Institutes of Health and Biotechnology Risk Assessment Program competitive grant number 2012-33522-19766 from the USDA—National Institute of Food and Agriculture. The United States Government may have certain rights in these inventions.

### TECHNICAL FIELD

The technical field relates to creation of genetically modified animals, for example, livestock animals with a knockout of a gametogenic gene.

### BACKGROUND

Livestock are conventionally created by sexual reproduction and raised to sexual maturity on farms, either with conventional pasturing and feeding practices, or by intensive farming practices, with the latter being increasingly common for swine. Sexual reproduction is a cost-effective and efficient process for the farmer.

### SUMMARY

An embodiment of the invention is a genetically modified livestock animal, the animal comprising a genetic modification to disrupt a target gene selectively involved in gametogenesis, wherein the disruption of the target gene prevents formation of functional gametes of the animal.

An embodiment of the invention is a process of preparing cells of a livestock animal comprising introducing, into an organism chosen from the group consisting of a livestock cell and a livestock embryo, an agent that specifically binds to a chromosomal target site of the cell and causes a double-stranded DNA break to disrupt a gene to selectively disrupt gametogenesis, with the agent being chosen from the group consisting of a targeted endonuclease, an RNA, and a recombinase fusion protein.

An embodiment of the invention is an in vitro cell comprising an agent that specifically binds to a chromosomal target site of the cell and causes a double-stranded DNA break to disrupt a gene to selectively disrupt gametogenesis, with the agent being chosen from the group consisting of a targeted endonuclease and a recombinase fusion protein.

An embodiment of the invention is a genetically modified livestock animal comprising a genomic modification to a Y chromosome, with the modification comprising an insertion, a deletion, or a substitution of one or more bases of the chromosome.

An embodiment of the invention is a genetically modified livestock animal, the animal comprising an exogenous gene on a chromosome, the gene being under control of a gene expression element that is selectively activated in gametogenesis.

An embodiment of the invention is a genetically modified animal comprising a genetically infertile male livestock animal that generates functional donor spermatozoa without production of functional native spermatozoa.

An embodiment of the invention is a genetically modified livestock animal, the animal comprising an exogenous gene on a chromosome, the gene expressing a factor that controls a gender of progeny of the animal, with said animal producing progeny of only one gender.

An embodiment of the invention is a herd comprising a plurality of said animals.

The following patent applications are hereby incorporated herein by reference for all purposes; in case of conflict, the specification is controlling: US 2010/0146655, US 2010/0105140, US 2011/0059160, and US 2011/0197290.

### BRIEF DESCRIPTION OF THE DRAWINGS

FIG. 1 is an illustration of a process of making and using animals that are genetically sterile to disseminate genes of a donor.

FIG. 2 is an illustration of a process to control gender and fertility by expression of factors by the Y-chromosome during gametogenesis.

FIG. 3A depicts a gene for disruption of gametogenesis with expression controlled by microRNA binding the 3′ UTR.

FIG. 3B depicts a microRNA for disruption of gametogenesis with expression controlled by microRNA binding the 3′ UTR and a late spermatogenesis promoter.

FIG. 4 depicts experimental results for modification of a vertebrate Y chromosome.

FIG. 5 is a montage of experimental results of Examples 6 and 7 showing CRISPR/Cas9 mediated HDR used to introgress the p65 S531P mutation from warthogs into conventional swine. Panel a) The S531P missense mutation. Panel b) SURVEYOR assay of transfected Landrace fibroblasts. Panels c and d) show RFLP analysis of cells sampled at days 3 and 10. The top and bottom rows of sequences in panel a are the guide RNA (gRNA) (P65_G1S having SEQ ID NO:1 and P65_G2A having SEQ ID NO:2). The second row is the wildtype (Wt) P65 sequence, SEQ ID NO:3. The third row is the HDR template, SEQ ID NO:4, used in the experiment. The left TALEN (SEQ ID NO:5) and right TALEN, (SEQ ID NO:6) are shown.

FIG. 6 is a montage of experiment results showing a comparison of TALENs and CRISPR/Cas9 mediated HDR at porcine APC. Panel a) depicts the APC14.2 TALENs and the gRNA sequence APC14.2 G1a relative to the wild type APC sequence. Below, the HDR oligo is shown which delivers a 4 bp insertion (underlined text) resulting in a novel HindIII site. Panel b) shows charts displaying RFLP and SURVEYOR assay results. The top row of panel a is the APC 14.2 TALENs sequence, SEQ ID NO:7. The second row is the wildtype APCS sequence, SEQ ID NO:8. The third row shows the gRNA sequence G1a, SEQ ID NO:9. The bottom sequence is the HDR template, SEQ ID NO:10.

FIG. 7 shows gene targeting of the vertebrate Y chromosome in two sites (AMELY and SRY) using TALENs and plasmid homology templates. Individual colonies are screened using a locus specific primer outside of the homology arms and a transgene specific primer within the homology template. The locus and orientation of the homology template is indicated above their corresponding wells and positive controls are indicated (+).

FIG. 8 is a table showing analysis results of Y-targeting in clones with TALENs and plasmid homology cassettes.

FIG. 9 is short homology targeting of Ubiquitin EGPF to 3 sites in the Y-chromosome. Primers for the 3′ junction of SRY also gave a non-specific banding pattern with and without TALENs.

FIG. 10 is a bar graph showing expression of the EGFP marker in cells treated with TALENs and short homology templates specific to AMELY and SRY sites.

FIG. 11 is a junction analysis of clones expressing the EGFP marker.

FIG. 12 is a montage of experimental results showing cloned pigs with HDR alleles of DAZL and APC. Panel A) is an RFLP analysis of cloned piglets derived from DAZL- and APC-modified landrace and Ossabaw fibroblasts, respectively. Panel B) is a sequence analysis confirming the presence of the HDR allele in three of eight DAZL founders, and in six of six APC founders. Blocking mutations, intended to inhibit re-cutting of the HDR allele, in the donor templates (HDR) are in boxes, and inserted bases are circled. The bold text in the top WT sequence indicates the TALEN-binding sites. Panel C) provides photographs of DAZL (Left) and APC (Right) founder animals. There are 14 rows of aligned sequences, with each row being a separate sequence numbered SEQ ID NO:11 to SEQ ID NO:24, respectively.

FIG. 13 is a photomicrographic montage of images showing that DAZL knockout (KO) pigs lack spermatogenesis and have no germ cells. Panel a) is H&E staining of DAZL KO seminiferous tubules from the inner portion of the testes that shows a complete absence of spermatogonia. Panel b) is H&E staining of DAZL KO seminiferous tubules from the outer portion of the testes, also showing a complete absence of spermatogonia. Panel c) uses a Ubiquitin carboxy-terminal hydrolase L1 (UCH-LI), a marker of spermatogonia present in wild type pig testes. In Panel d) UCH-LI is absent in DAZL KO testes, indicating an absence of spermatogonia. In Panel e) acetylated a-tubulin is present in the seminiferous tubules of wild type pig testes, indicating the presence of spermatogonia. In Panel f) DAZL KO pig seminiferous tubules are negative for acetylated a-tubulin demonstrating a lack of germ cells in these animals.

### DETAILED DESCRIPTION

Embodiments are set forth herein to make and use genetically sterile animals, or animals that are capable of producing only one gender of progeny. The availability of genetically sterile animals and facile techniques for their creation, as set forth herein, provides new methods of, and new opportunities in, production of genetically modified animals and conventional livestock. Some embodiments involve placing donor tissue into genetically sterile recipient males so that the recipient males produce donor sperm and can be used as studs to make progeny of the donor animals. This technique allows the use of sexual reproduction to disseminate desirable genetic traits, including genetically engineered traits.

Other embodiments are used to protect valuable traits: for instance, an animal that is bred and/or is genetically modified to have one or more desirable traits can also be modified so that it is sterile, or has progeny of only one sex, thus ensuring that these valuable traits will not be misappropriated or escape containment.

Conventional animal production and genetically modified animal production processes emphasize fertility and viability. Livestock reproductive inefficiencies have a large, negative impact on livestock production. Despite an increasing number of techniques that can be used to increase reproductive success, losses in the reproductive cycle are common. Sophisticated techniques, including cloning, are known, but are much less efficient than sexual reproduction and are not suited to mass production of livestock. In an animal with highly prized genetics, artificial insemination or embryo-transfer may sometimes be used to maximize the transmission of its genes to progeny. Cloning techniques such as somatic cell nuclear transfer or chromatin transfer have a low efficiency that is not comparable to sexual reproduction and is not suitable for routine production of genetically modified animals. Cloning using embryonic stem cells, which is called Nuclear Transfer-derived Embryonic Stem Cell (NTESC) is not presently possible for livestock since derivation of livestock embryonic stem cells has been unsuccessful to date.

The use of genetic engineering to create genetically modified livestock will accelerate the creation of livestock with desirable traits. Traditional livestock breeding is an expensive and time consuming process that involves careful selection of genetic traits and lengthy waits for generational reproduction. Even with careful trait selection, the variations of sexual reproduction present a considerable challenge in cultivating and passing on a desirable trait combinations.

Presented herein are embodiments for animal reproduction that allow for rapid dissemination of desirable genetic traits, as well as for protection of the proprietary control and containment of the traits. Embodiments include the production of genetically and genomically sterile animals that can serve as hosts for donated genetic material. Sexual intercourse by the host will lead to reproduction of the donor's genetic material. A group of genetically sterile animals can be used to widely disseminate identical germplasma from a single donor by sexual reproduction so that many donor progeny may be rapidly generated. Embodiments include donors that are modified to produce only one gender of animal so that users receiving the animals will not be able to misappropriate the animals with the traits, nor lose containment of them.

A genomically sterile animal is consistently sterile, meaning that it genetically cannot produce progeny. The term sterile, in this context, means unable to use sexual reproduction to produce progeny with its own genetic makeup. Thus an animal that produces progeny of a donor animal is referred to as sterile although it is active in creating functional gametes for another animal. In some cases, the sterile animal produces its own gametes that can be removed and used in an artificial reproductive process; for example, a host animal that makes immotile sperm can be propagated by intracytoplasmic sperm injection (ICSI), or a host animal can be propagated by cloning. A functional gamete is a gamete that is useful for successful sexual reproduction. A genomically sterile animal can be prepared that hosts gametogenesis for donor gametogenic cells. The term gametogenesis means the production of haploid sex cells (ova and spermatozoa) that each carry one-half the genetic compliment from the germ line of each parent. The production of spermatozoa is spermatogenesis. The fusion of spermatozoa and ova during fertilization results in a zygote cell that has a diploid genome. The term gametogenic cell refers to a progenitor to an ovum or sperm, typically a germ cell, oogonial cell, or a spermatogonial cell.

Embodiments of the invention include genomically sterile animals that have a genetic modification to a chromosome that prevents gametogenesis or spermatogenesis in that animal. The chromosome may be an X chromosome, a Y chromosome, or an autosome. The modification may include a disruption of an existing gene. The disruption may be created by altering an existing chromosomal gene so that it cannot be expressed, or by genetically expressing factors that will inhibit the transcription or translation of a gene. Some of the techniques used to make genetically sterile animals can also be applied to make animals that produce only male or female progeny, having transmitting their genetics or the genetics of a donor.

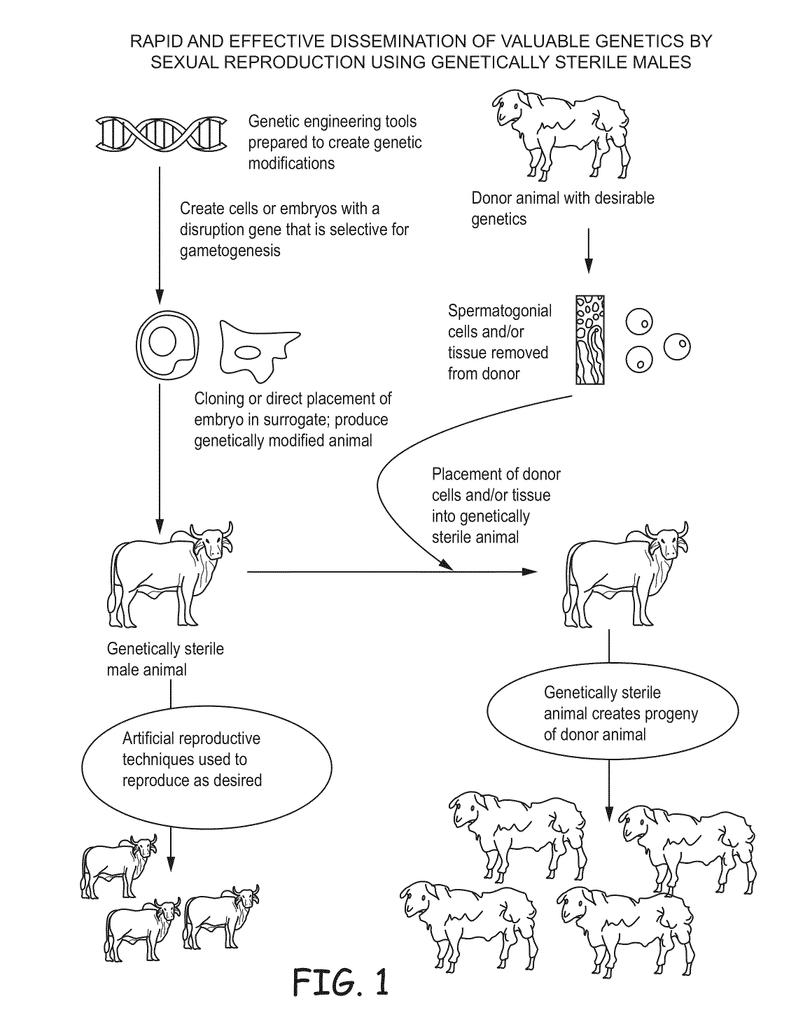

An embodiment of a genetically sterile animal comprising a genomic disruption of a gene encoding a factor selectively involved in gametogenesis, wherein the animal is sterile when hemizygous or homozygous for the disruption is illustrated in FIG. 1. The terms disruption and inactivation are used interchangeably herein. A genetic modification is made to cells or embryos to inactivate a gene that is selective for spermatozoa activity. One process of genetic modification involves introduction of mRNA for a TALEN pair that specifically binds and breaks the gene. An animal is cloned from the cells into an embryo, or a modified embryo is directly raised in a surrogate mother. The animal may be a livestock animal or other animal. The spermatozoa activity that is disrupted is essential for fertility but is not otherwise essential to the animal. The animal is thus sterile because it cannot sexually reproduce: however, ARTs may be used to create progeny from the modified sperm. A donor animal that has desirable genetic traits (as a result of breeding and/or genetic engineering) is selected. The illustration shows a double muscled Belgian Blue bull donor. Spermatogonial cells and/or spermatogonial tissue is taken from the donor and implanted into the recipient sterile animal. Implantation at the seminiferous tubules allows for the donor cells and tissue to reproduce to make functional sperm (Brinster and Avarbock, Spermatogenesis following male germ-cell transplantation. PNAS. 91:11298-11302, 1994). The genetically sterile animal is thus made into a tool for dissemination of the donor's genetics, and mating the animal with multiple females provides for a rapid spread of desirable genetic traits.

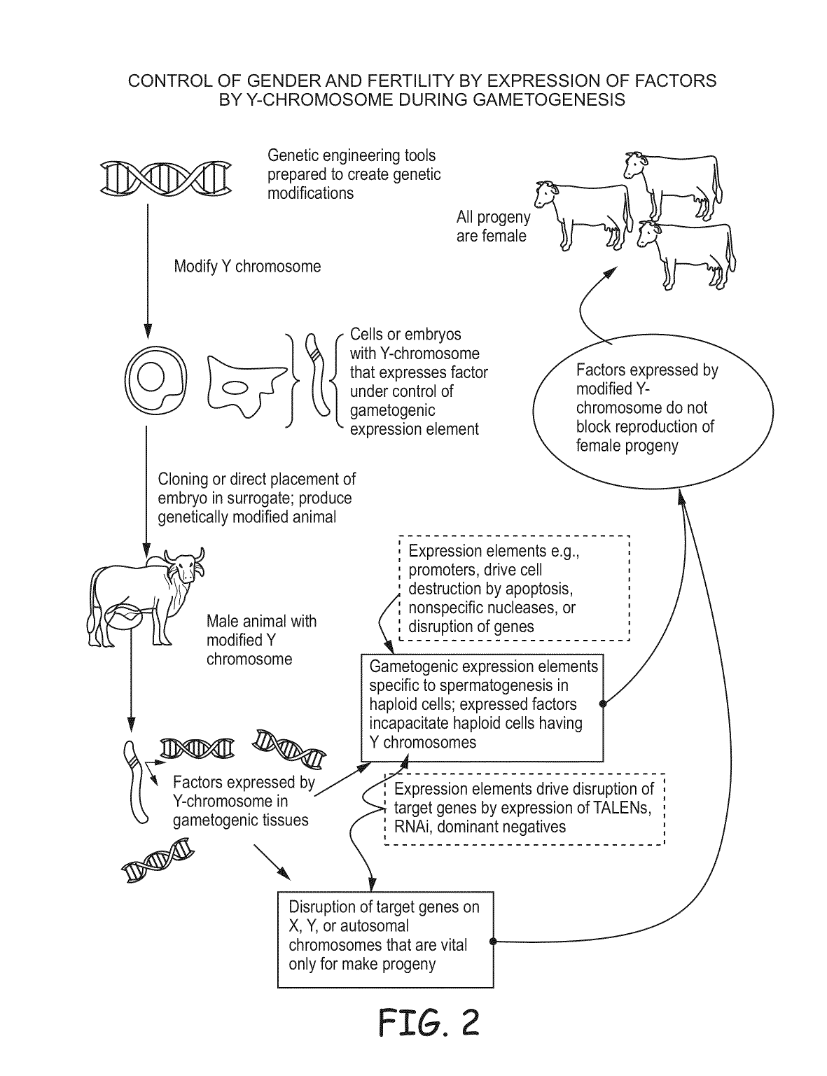

An embodiment of a genetically modified livestock animal, the animal comprising cells that comprise a chromosome that comprises an exogenous gene under control of a promoter selectively activated in gametogenesis, is illustrated in FIG. 2. As explained for FIG. 1, an animal is created by genetic modification of a cell or embryo. In the embodiment in the Figure, the chromosome is a Y chromosome. The factor that is expressed by the exogenous gene is under control of a promoter selective for gametogenesis, or for a stage of spermatogenesis. The factor may disrupt a target gene such as a gene that is necessary for development of a male animal but is not necessary for the development of a female, or vice versa. Or the gene may be placed under the transcriptional control of a promoter selectively activated in gametogenesis or spermatogenesis, with the factor being disruptive to, or fatal to, a cell to thereby prevent development of or to destroy, only male gametes, whereby only female offspring are produced, or vice versa. The promoter may be active inside the cell or in tissue specific for gametogenesis, spermatogenesis, or oogenesis, for instance tissue selected from the group consisting of testes, seminiferous tubules, or epidydimus, or in the case of oogenesis the ovary, follicle, oocyte, granulosa cells or corpus luteum. Promoters for female gametogenesis include, for example, Nobox, Oct4, Bmp15, Gdf9=FecB, Oogenesin1 and Oogenesin2.

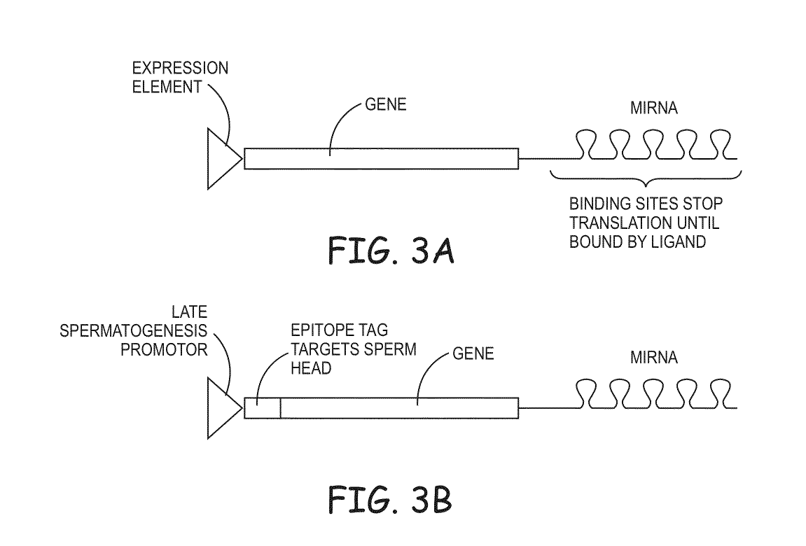

FIG. 3 describes a further modification to above where exogenous factor is also under the control of microRNAs binding sequences placed into the 3′ UTR, such that the factor is not translated in tissues where the microRNA is expressed but in tissues where the microRNA is not expressed, for instance tissue selected from the group consisting of testes, seminiferous tubules, or epidydimus, the factor would be translated. This approach could use a ubiquitous or tissue specific promoter. In a second embodiment, the 3′ UTR would include microRNA sequences that target a gene necessary for development spermatozoa or gametes. An embodiment is a genetically modified livestock animal, the animal comprising cells that comprise a chromosome that comprises an exogenous gene expression element that when expressed in the context of an mRNA can serve target for the binding of ligands that attenuate transcription, degrade/stabilize mRNA, localize mRNA, or can suppress or activate translation. Ligands can include RNA-binding proteins (which do and don't also contain protein binding domains) such as those in the RNA-binding Proteins Database (RBPDB), including but not restricted to proteins that contain a Nucleic Acid recognition domain, RNA Recognition Motif (RRM), K-Homology Domain (KH domain), Zinc Finger domain, TALE-like Repeats, Pumilio and FBF homology (PUF) repeats, or pentatricopeptide repeat (PPR) proteins. Ligands can also include Regulatory RNAs such as transfer RNAs, Antisense RNA, CRISPR RNA, Long noncoding RNA, MicroRNA, Piwi-interacting RNA, Small interfering RNA, Trans-acting siRNA, Repeat associated siRNA. Expression of either the target or the regulatory ligand can be selectively activated or repressed in gametogenesis, oogenesis or spermatogenesis.

### Genes for Modification

Genes in one livestock species consistently have orthologs in other livestock species, as well as in humans and mice. Humans and mice genes consistently have orthologs in livestock, particularly among cows, pigs, sheep, goats, chicken, and rabbits: Genetic orthologs between these species and fish is often consistent, depending upon the gene's function. Biologists are familiar with processes for finding gene orthologs so genes may be described herein in terms of one of the species without listing orthologs. Embodiments describing the disruption of a gene thus include disruption of orthologs that have the same or different names in other species. There are general genetic databases as well as databases that are specialized to identification of genetic orthologs. Genes for disruption include genes selective for gametogenesis, specifically, spermatogenesis. Motifs for disabling spermatogenesis without destruction of the sperm's gamete are to interfere with the sperm's motility, acrosome fusion, or syngamy. Target genes may include those chosen from the group consisting of TENR, ADAM1a, ADAM2, ADAM, alpha4, ATP2B4 gene, CatSper1, CatSper2, CatSper3, Catsper4, CatSperbeta, CatSpergamma, CatSperdelta, KCNU1, DNAH8, Clamegin, Complexin-I, Sertoli cell androgen receptor, Gasz, Ra175, Cib1, Cnot7, Zmynd15, CKs2, PIWIL4, PIWIL2, and Smcp.

Embodiments of genes that may be disrupted to interfere with sperm motility are TENR (Connolly C M; Dearth A T; Braun R E Disruption of murine Tenr results in teratospermia and male infertility. Dev Biol. 278(1):13-21, 2005); ADAM1a (Nishimura H; Kim E; Nakanishi T; Baba T Possible function of the ADAM1a/ADAM2 Fertilin complex in the appearance of ADAM3 on the sperm surface. J Biol Chem. 279(33):34957-62, 2004); and ADAM3 (Shamsadin R; Adham I M; Nayernia K; Heinlein U A; Oberwinkler H; Engel W Male mice deficient for germ-cell cyritestin are infertile. J. Biol. Reprod. 61(6):1445-51, 1999). A knockout of alpha4 (Atp1a4, ATPase, Na+/K+ transporting, alpha 4 polypeptide) makes animals that are completely sterile and results in severe reduction in sperm motility (Jimenez T; McDermott J P; Sanchez G; Blanco G Na,K-ATPase alpha4 isoform is essential for sperm fertility. Proc. Natl. Acad. Sci. USA 108(2):644-649, 2011). Male mice with a targeted gene deletion of isoform 4 of plasma membrane calcium/calmodulin-dependent calcium ATPase (PMCA4, encoded by ATP2B4 gene), which is highly enriched in the sperm tail, are infertile due to severely impaired sperm motility. Schuh K; Cartwright E J; Jankevics E; Bundschu K; Liebermann J; Williams J C; Armesilla A L; Emerson M; Oceandy D; Knobeloch K P; Neyses L Plasma membrane Ca2+ATPase 4 is required for sperm motility and male fertility. J. Biol. Chem. 279(27):28220-28226, 2004).

Embodiments of genes that may be disrupted to interfere with sperm acrosome fusion and/or capacitation are: ADAM2 or ADAM3, (Nishimura H; Cho C; Branciforte D R; Myles D G; Primakoff P Analysis of loss of adhesive function in sperm lacking cyritestin or fertilin beta. Dev. Biol. 233(1):204-213, 2001). A knockout of alpha4 (referenced above) also results in spermatozoa from these mice are unable of fertilizing eggs in vitro. Genes in the CatSper family may be selectively disrupted to create male animals that are unable to create offspring by sexual reproduction. CATSPER family genes provide transmembrane calcium channel proteins that are embedded in the membrane of sperm cells. Calcium cations are required for hyperactivation, which is necessary for the sperm to push through the membrane of the egg cell during fertilization. A CatSper gene or a subunit of a channel encoded by Catsper may be disrupted to create infertile males. Males disrupted for CatSper2 are completely infertile and their sperm are unable to penetrate the egg (Quill T A; Sugden S A; Rossi K L; Doolittle L K; Hammer R E; Garbers D L Hyperactivated sperm motility driven by CatSper2 is required for fertilization. Proc. Natl. Acad. Sci. USA 100(25):14869-14874, 2003). Disruption of Catsper2 or CatSper3 or Catsper4 has a similar effect (Qi H; Moran M M; Navarro B; Chong J A; Krapivinsky G; Krapivinsky L; Kirichok Y; Ramsey I S; Quill T A; Clapman D E All four CatSper ion channel proteins are required for male fertility and sperm cell hyperactivated motility Proc. Natl. Acad. Sci. USA 2007). Clamegin (Clgn) disruption in mice causes sperm to be unable to penetrate the zona pellucida (Ikawa M; Wada I; Kominami K; Watanabe D; Toshimori K; Nishimune Y; Okabe M The putative chaperone calmegin is required for sperm fertility. Nature 387(6633):607-611, 1997). Complexin-I (Cplx1) deficient sperm are subfertile due to faulty zona pellucida penetration. (Zhao L; Reim K; Miller D J Complexin-I-deficient sperm are subfertile due to a defect in zona pellucida penetration. Reproduction 136(3):323-334, 2008). Disruption of potassium channel Kcnu1 (NCBI Gene ID 157855, also known as Kcnma3, Slo3, KCa5, KCa5.1, KCNMC1) also creates males with sperm that are unable to undergo capacitation such that there is no fertilization. DNAH8 (Gene ID: 1769, also known as hdhc9) disruption results in immotile sperm by interference with flagellar machinery thereby interfering with movement.

Vasa is an RNA binding protein with an RNA dependant helicase. The vasa gene is essential for germ cell development Vasa-null animals have been generated in Drosophila, Caenorhabditis elegans and mice by gene knockout, by reduction of Vasa mRNA by RNA interference (RNAi) and by Vasa protein reduction by antisense morpholino treatment (knockdown), Gustafson and Wessel, Bioessays 32:626-637, 2010 The human vasa gene is Ddx4, see Castrillon et al., PNAS 97(17):9585-9590. In animal models, a null mutation that removes the entire vasa coding region results in female sterility with severe defects in oogenesis, including abnormal germ-line differentiation and oocyte determination. Females homozygous for partial loss-of-function alleles produce eggs that can be fertilized, but the resulting embryos lack germ cells. Therefore, vasa function is not only required during gametogenesis in the adult but is also essential for the specification of the germ cell lineage during embryogenesis (Castrillon et al.). Male mice homozygous for a targeted mutation of the mouse vasa ortholog Mvh are sterile and exhibit severe defects in spermatogenesis, whereas homozygous females are fertile. Embodiments of the invention include livestock animals with disrupted vasa genes as well as vasa genes disruptable under induced control.

Some genes, when disrupted, selectively interfere with spermatogenesis and prevent, or destroy, formation of a gamete, for instance genes in the DAZ family, DAZL, and DAZ1. DAZ1 is selective for gametogenesis, specifically, spermatogenesis, with disruption causing no sperm to form. DAZ1 is on the Y-chromosome. Alpha1b encodes for the alpha1b adrenergic receptor and knockouts can be hypofertile or lack spermatogenesis altogether (Mhaouty-Kodja S; Lozach A; Habert R; Tanneux M; Guigon C; Brailly-Tabard S; Maltier J P; Legrand-Maltier C Fertility and spermatogenesis are altered in alpha1b-adrenergic receptor knockout male mice. J Endocrinol 195(2):281-292, 2007). Disruption of the X-chromosome's Sertoli cell androgen receptor (Ar) at the AR DNA-binding domain (AR-DBD) showed that the AR-DBD is essential for SC function and postmeiotic spermatogenesis, and produced infertile males exhibiting spermatogenic arrest, despite normal Sertoli cell numbers (Lim P; Robson M; Spaliviero J; McTavish K J; Jimenez M; Zajac J D; Handelsman D J; Allan C M Sertoli cell androgen receptor DNA binding domain is essential for the completion of spermatogenesis. Endocrinology 150(10):4755-4765, 2009; see also Krutskikh A; De Gendt K; Sharp V; Verhoeven G; Poutanen M; Huhtaniemi I Targeted inactivation of the androgen receptor gene in murine proximal epididymis causes epithelial hypotrophy and obstructive azoospermia. Endocrinology 152(2):689-696, 2011). A knockout of Gasz in mice results in a zygotene-pachytene spermatocyte block and male sterility defect observed (Ma L; Buchold G M; Greenbaum M P; Roy A; Burns K H; Zhu H; Han D Y; Harris R A; Coarfa C; Gunaratne P H; Yan W; Matzuk M M GASZ is essential for male meiosis and suppression of retrotransposon expression in the male germline. PLoS Genet 5(9):e1000635, 2009). Male mice lacking both alleles of Ra175 (Ra175−/−) were infertile and showed oligo-astheno-teratozoospermia; almost no mature motile spermatozoa were found in the epididymis (Fujita E; Kouroku Y; Ozeki S; Tanabe Y; Toyama Y; Maekawa M; Kojima N; Senoo H; Toshimori K; Momoi T Oligo-astheno-teratozoospennia in mice lacking RA175/TSLC1/SynCAM/IGSF4A, a cell adhesion molecule in the immunoglobulin superfamily. Mol. Cell Biol. 26(2):718-726, 2006). Disruption of Cib1 made the males are sterile due to disruption of the haploid phase of spermatogenesis (Yuan W; Leisner T M; McFadden A W; Clark S; Hiller S; Maeda N; O'brien DA; Parise L V CIB1 Is Essential for Mouse Spermatogenesis. Mol. Cell Biol. 26(22):8507-8514, 2006). Cnot7-disrupted males are sterile owing to oligo-astheno-teratozoospermia (Nakamura T; Yao R; Ogawa T; Suzuki T; Ito C; Tsunekawa N; Inoue K; Ajima R; Miyasaka T; Yoshida Y; Ogura A; Toshimori K; Noce T; Yamamoto T; Noda T Oligo-astheno-teratozoospermia in mice lacking Cnot7, a regulator of retinoid X receptor beta. Nat Genet 36(5):528-33, 2004). Disruption of Cul4A by genetic knockout or by expression of a dominant negative caused infertility with a defect in spermatogenesis (Kopanja D; Roy N; Stoyanova T; Hess R A; Bagchi S; Raychaudhuri P Cul4A is essential for spermatogenesis and male fertility. Dev Biol. 352(2):278-287, 2011). ZMYND15 acts as a histone deacetylase-dependent transcriptional repressor and controls normal temporal expression of haploid cell genes during spermiogenesis. Inactivation of Zmynd15 results in early activation of transcription of numerous important haploid genes including Prm1, Tnp1, Spem1, and Catpser3; depletion of late spermatids; and male infertility (Yan W; Si Y; Slaymaker S; Li J; Zheng H; Young D L; Aslanian A; Saunders L; Verdin E; Charo I F Zmynd15 encodes a histone deacetylase-dependent transcriptional repressor essential for spermiogenesis and male fertility. J Biol. Chem. 285(41):31418-31426, 2010).

Other genes disrupt all gametogenesis for both males and females so that disruption of these genes in animal lines produces sterile offspring. One such gene is CKs2. Mice lacking Cks2, were viable but sterile in both sexes. Sterility is due to failure of both male and female germ cells to progress past the first meiotic metaphase.

Some genes are disrupted in combination to produce one or more effects that cause infertility, for instance, combinations of: Acr/H1.1/Smcp, Acr/Tnp2/Smcp, Tnp2/H1.1/Smcp, Acr/H1t/Smcp, Tnp2/Hlt/Smcp (Nayernia K; Drabent B; Meinhardt A; Adham I M; Schwandt I; Muller C; Sancken U; Kleene K C; Engel W Triple knockouts reveal gene interactions affecting fertility of male mice. Mol. Reprod. Dev 70(4):406-416, 2005). Embodiments include a first line of animals with a knockout of the indicated gene combinations and/or subcombinations.

### Genetically Modified Animals

Animals may be made that are mono-allelic or bi-allelic for a chromosomal modification, using methods that either leave a marker in place, allow for it to be bred out of an animal, or by methods that do not place a marker in the animal. For instance, the inventors have used methods of homologous dependent recombination (HDR) to make changes to, or insertion of exogenous genes into, chromosomes of animals. Tools such as TALENs and recombinase fusion proteins, as well as conventional methods, are discussed elsewhere herein. Some of the experimental data supporting genetic modifications disclosed herein is summarized as follows. The inventors have previously demonstrated exceptional cloning efficiency when cloning from polygenic populations of modified cells, and advocated for this approach to avoid variation in cloning efficiency by somatic cell nuclear transfer (SCNT) for isolated colonies (Carlson et al., 2011). Additionally, however, TALEN-mediated genome modification, as well as modification by recombinase fusion molecules, provides for a bi-allelic alteration to be accomplished in a single generation. For example, an animal homozygous for a knocked-out gene may be made by SCNT and without inbreeding to produce homozygosity. Gestation length and maturation to reproduction age for livestock such as pigs and cattle is a significant barrier to research and to production. For example, generation of a homozygous knockout from heterozygous mutant cells (both sexes) by cloning and breeding would require 16 and 30 months for pigs and cattle respectively. Some have reduced this burden with sequential cycles of genetic modification and SCNT (Kuroiwa et al., 2004) however, this is both technically challenging and cost prohibitive. The ability to routinely generate bi-allelic KO cells prior to SCNT is a significant advancement in large animal genetic engineering. Bi-allelic knockout has been achieved in immortal cells lines using other processes such as ZFN and dilution cloning (Liu et al., 2010). Another group recently demonstrated bi-allelic KO of porcine GGTA1 using commercial ZFN reagents (Hauschild et al., 2011) where bi-allelic null cells could be enriched by FACS for the absence of a GGTA1-dependent surface epitope. While these studies demonstrate certain useful concepts, they do not show that animals or livestock could be modified because simple clonal dilution is generally not feasible for primary fibroblast isolates (fibroblasts grow poorly at low density) and biological enrichment for null cells is not available for the majority of genes.

The inventors have previously shown that transgenic primary fibroblasts can be effectively expanded and isolated as colonies when plated with non-transgenic fibroblasts at densities greater than 150 cells/cm2 and subjected to drug selection using a transposon co-selection technique (Carlson et al., 2011, U.S. Pub. No. 2011/0197290). It was further shown (see U.S. Ser. No. 13/404,662 filed Feb. 24, 2012) that puromycin resistant colonies were isolated for cells treated with six TALEN pairs and evaluated their genotypes by SURVEYOR assay or direct sequencing of PCR products spanning the target site. In general, the proportion of indel positive clones was similar to predictions made based on day 3 modification levels. Bi-allelic KO clones were identified for 5 of 6 TALEN pairs, occurring in up to 35% of indel positive cells. Notably, the frequency of bi-allelic KO clones for the majority of TALEN pairs exceeds what would be predicted if the cleavage of each chromosome is treated as an independent event.

TALEN-induced homologous recombination eliminates the need for linked selection markers. TALENs may be used to precisely transfer specific alleles into a livestock genome by homology dependent repair (HDR). In a pilot study, a specific 1 lbp deletion (the Belgian Blue allele) (Grobet et al., 1997; Kambadur et al., 1997) was introduced into the bovine GDF8 locus (see U.S. Ser. No. 13/404,662 filed Feb. 24, 2012). When transfected alone, the btGDF8.1 TALEN pair cleaved up to 16% of chromosomes at the target locus. Co-transfection with a supercoiled homologous DNA repair template harboring the 11 bp deletion resulted in a gene conversion frequency (HDR) of up to 5% at day 3 without selection for the desired event. Gene conversion was identified in 1.4% of isolated colonies that were screened. These results demonstrated that TALENs can be used to effectively induce HDR without the aid of a linked selection marker. Example 1 provides experimental data showing that a Y-chromosome, or other chromosomes, may be genetically altered by using, for instance, TALENs. TALENs are discussed in more detail elsewhere herein.

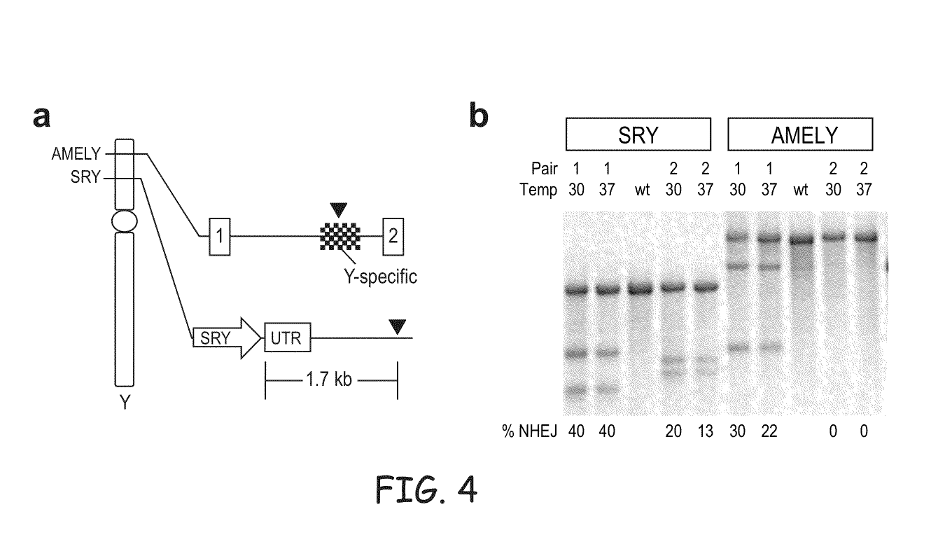

Example 1, see FIG. 4, describes TALENs directed to targets at the Y chromosome. Three TALENs pairs showed activity. Accordingly, cells can be made with indels on the Y chromosome, and animals from the cells. Example 2 provides methods for a TALEN-mediated genome modification to achieve a bi-allelic knockout in single generation. Gestation length and maturation to reproduction age for pigs and cattle is significant; for example, generation of a homozygous knockout from heterozygous mutant cells (both sexes) by cloning and breeding would require 16 and 30 months for pigs and cattle respectively. Bi-allelic knockout has been achieved in immortal cells lines using ZFN and dilution cloning (Liu et al., 2010). Another group recently demonstrated bi-allelic knockout of porcine GGTA1 using commercial ZFN reagents (Hauschild et al., 2011) where bi-allelic null cells could be enriched by FACS for the absence of a GGTA1-dependent surface epitope. While these other studies are useful, they use simple clonal dilution. Such processes are not feasible for the majority of primary fibroblast isolates and biological enrichment for null cells is not available for the majority of genes. In Example 2, however, primary cells were used, based on a method that permits expansion of individual colonies to screen for bi-allelic knockout. Example 3 demonstrates an alternative method of modifying cells useful for making cloned animals. Examples 4 demonstrates other methods of making cells for cloning, specifically, methods involving single-stranded oligonucleotides as HDR templates. Example 5 uses the single-stranded oligonucleotide processes to move genes from one species to another in an efficient process that is free of markers.

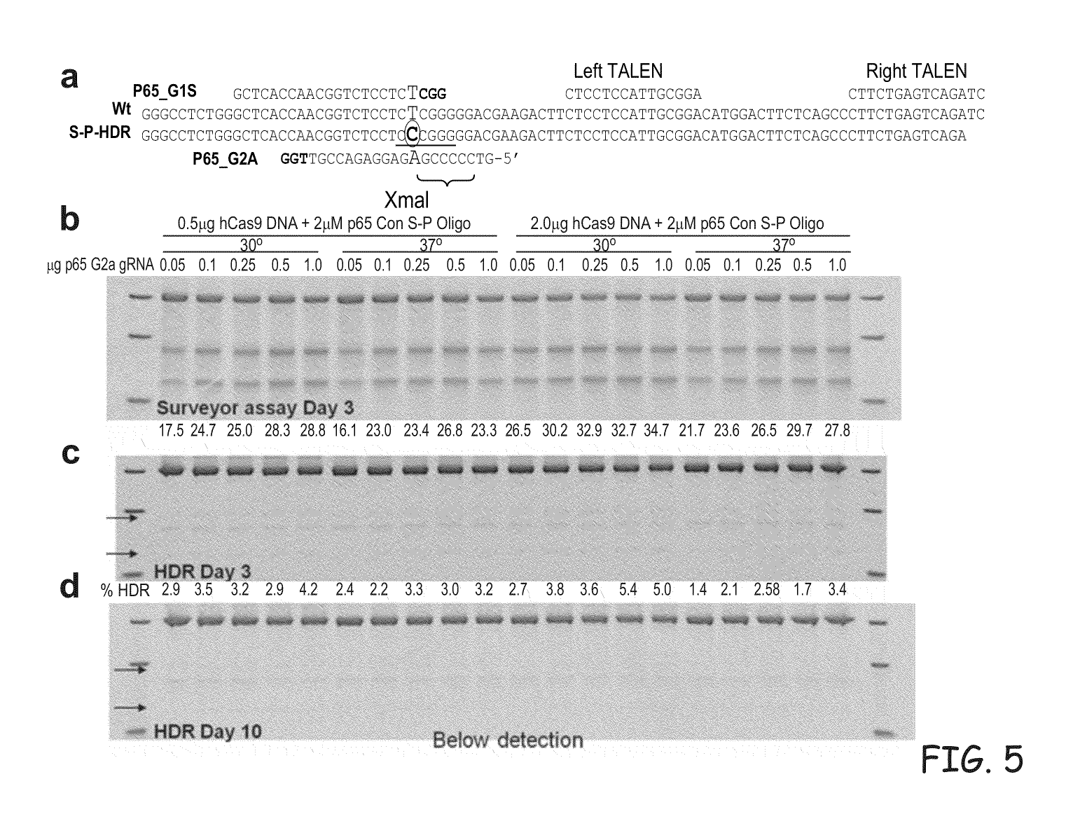

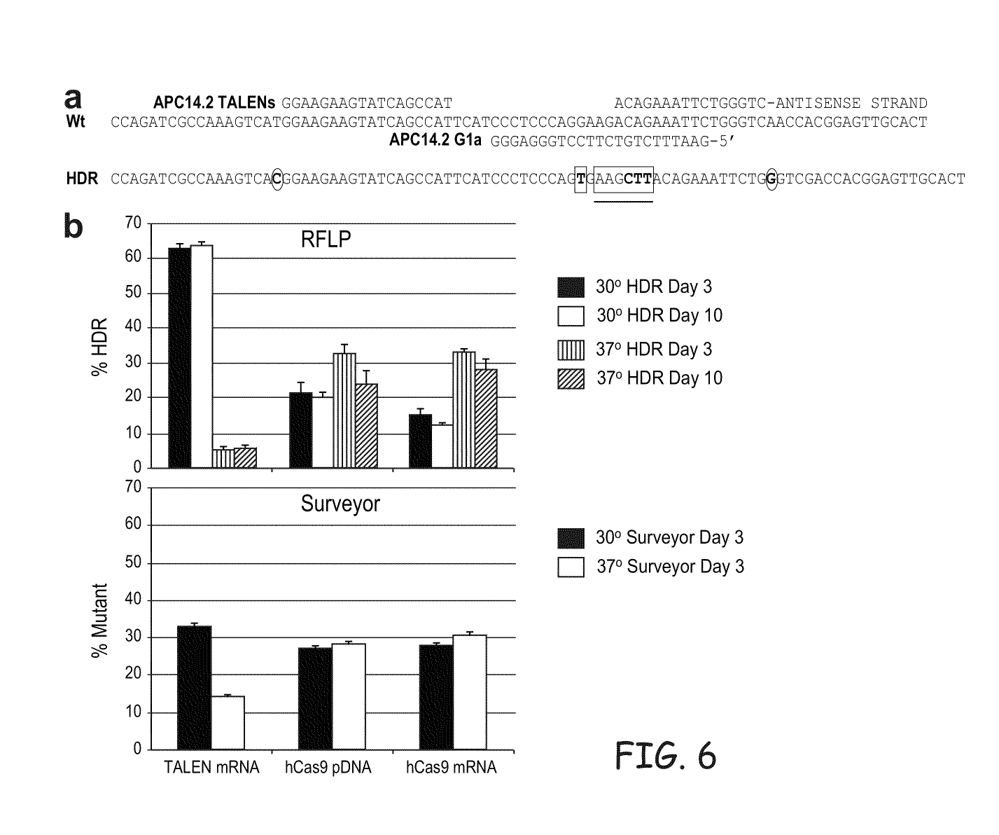

Examples 6-8 describe Cas9/CRISPR nuclease editing of genes. Examples 7 and 8 are Cas9/CRISPR results, showing efficient production of double stranded breaks at the intended site. Such breaks provide opportunities for gene editing by HDR template repair processes. CRISPR/Cas9-mediated HDR was lower than 6 percent at day-3 and below detection at day-10 (FIG. 5). Analysis of CRISPR/Cas9 induced targeting at a second locus, ssAPC14.2, was much more efficient, but still did not reach the level of HDR induced by TALENs at this site, about 30% versus 60% (FIG. 6). Cas9/CRISPR was an effective tool, as shown by these experiments.

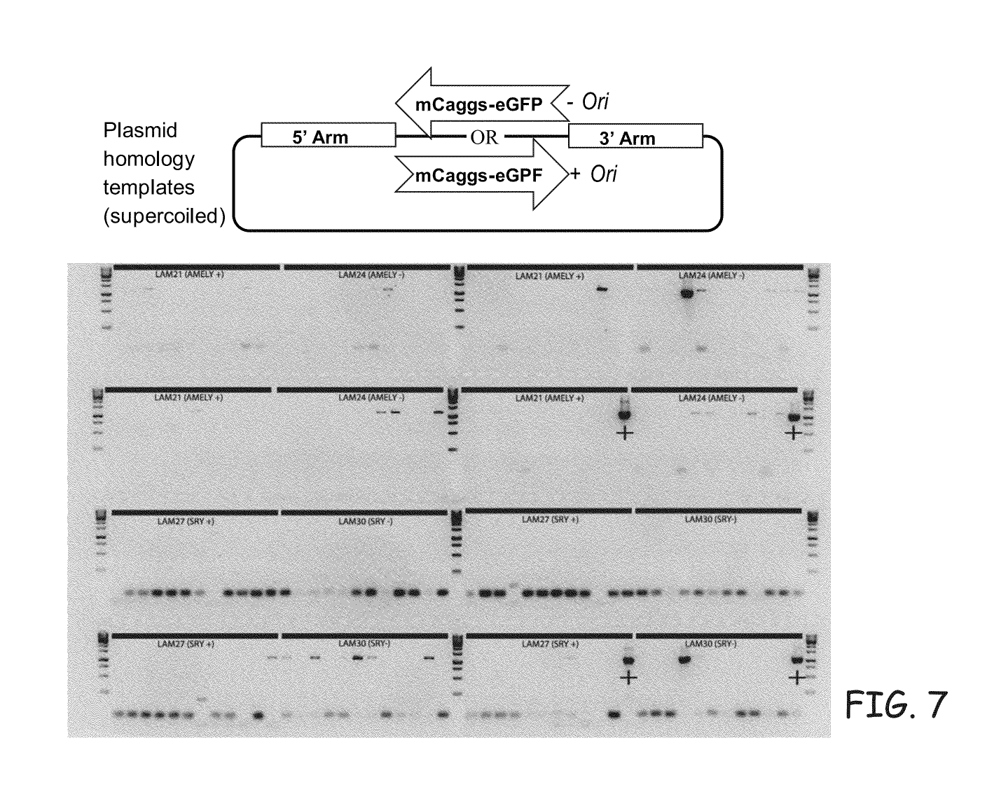

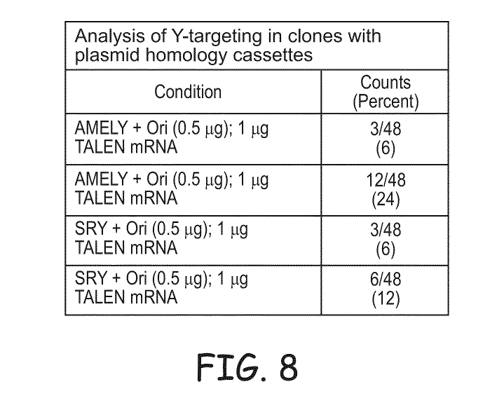

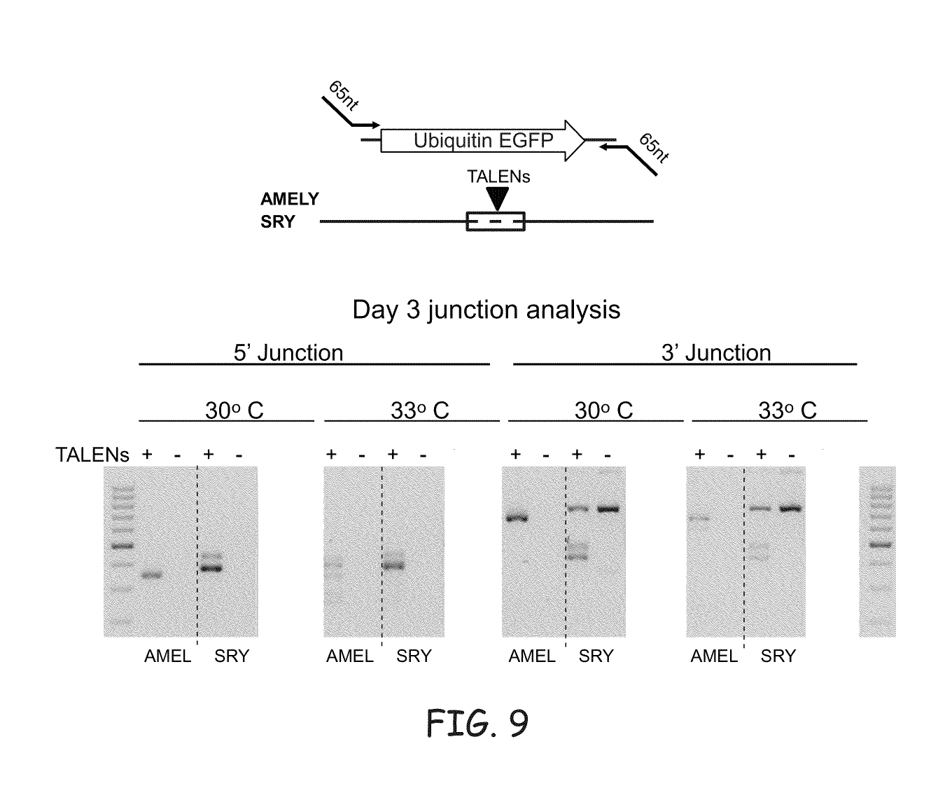

Examples 9 and 10 describe targeting of the Y-chromosome with either a plasmid cassette (FIGS. 7 and 8) or with a linear short homology template (FIGS. 9-11). Both techniques used TALENs to create a double strand break at the intended targeting site and homology templates directed the gene of interest to the target location. The efficiency was between 1 and 24% with both methods being effective.

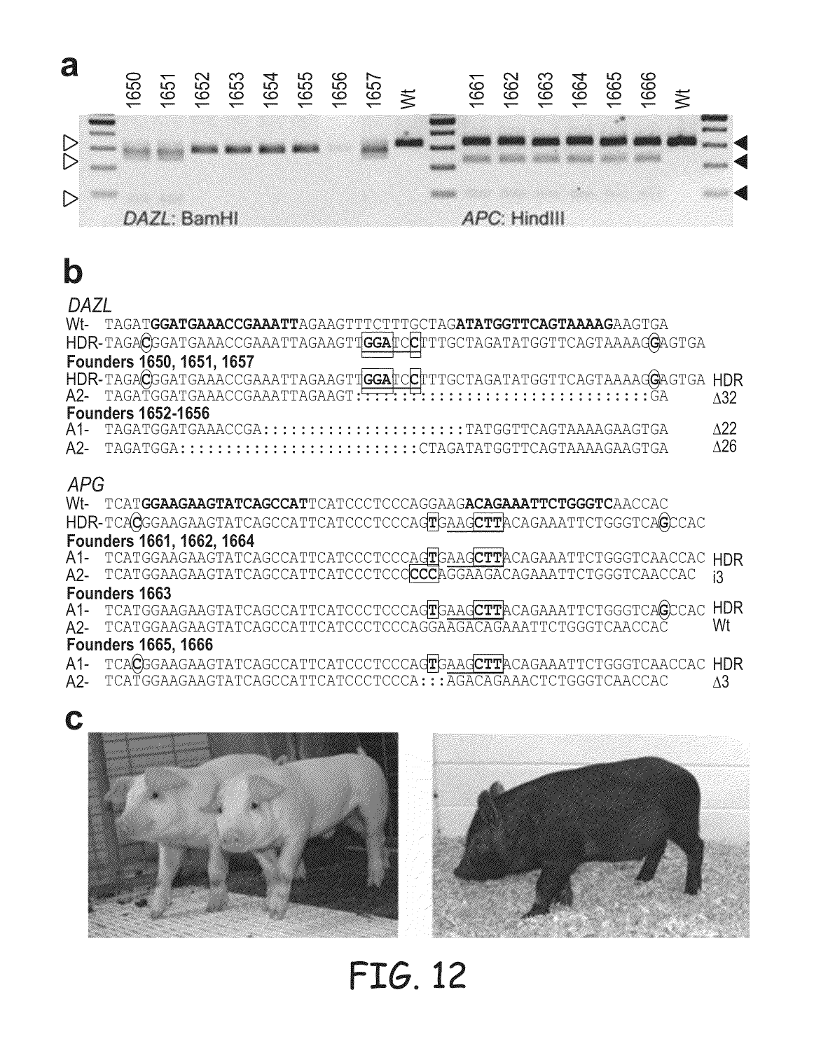

Example 11, see FIG. 12, describes processes for making animals with a disrupted DAZL gene or disrupted APC gene. The DAZL knockouts create sterile animals. As explained herein, the animals can be treated with donor cells or tissue to produce gametes that distribute the genetics of the donor animal by sexual reproduction. DAZL knockout pigs were made with these techniques. These are described in Example 12.

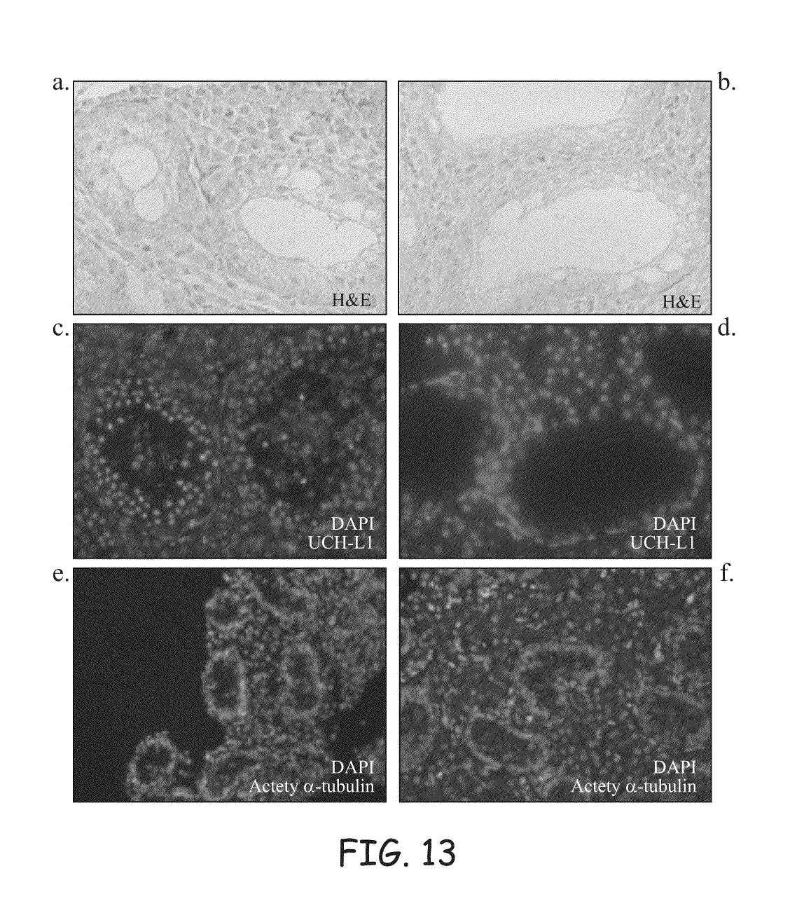

Example 12, see FIG. 13. Describes the sterile and germ cell free phenotype of the DAZL KO animals. Animals or cells edited to disrupt the DAZL gene are useful as a model for studying the restoration of human fertility by germ cell transplantation, or for the production of genetically modified offspring by transfer of genetically modified germline cells. Now that this process has been established for DAZL, it can be recreated with other genes that disrupt gametogenesis.

Experimental results indicated that targeted nuclease systems were effectively cutting dsDNA at the intended cognate sites. Targeted nuclease systems include a motif that binds to the cognate DNA, either by protein-to-DNA binding, or by nucleic acid-to-DNA binding. The efficiencies reported herein are significant. The inventors have disclosed further techniques elsewhere that further increase these efficiencies.

Embodiments of the invention include a method of making a genetically modified animal, said method comprising exposing embryos or cells to a vector or an mRNA encoding a targeting nuclease (e.g., meganuclease, zinc finger, TALENs, guided RNAs, recombinase fusion molecules), with the targeting nuclease specifically binding to a target chromosomal site in the embryos or cells to create a change to a cellular chromosome, cloning the cells in a surrogate mother or implanting the embryos in a surrogate mother, with the surrogate mother thereby gestating an animal that is genetically modified without a reporter gene and only at the targeted chromosomal site. The targeted site may be one as set forth herein, e.g., the various genes described herein.

Production of Biomedical Model Animals with Gene-Edited Alleles

Two gene-edited loci in the porcine genome were selected to carry through to live animals—APC and DAZL. Mutations in the adenomatous polyposis coli (APC) gene are not only responsible for familial adenomatous polyposis (FAP), but also play a rate-limiting role in a majority of sporadic colorectal cancers. DAZL (deleted in azoospermia-like) is an RNA binding protein that is important for germ cell differentiation in vertebrates. The DAZL gene is connected to fertility, and is useful for infertility models as well as spermatogenesis arrest. Colonies with HDR-edited alleles of DAZL or APC for were pooled for cloning by chromatin transfer. Each pool yielded two pregnancies from three transfers, of which one pregnancy each was carried to term. A total of eight piglets were born from DAZL modified cells, each of which reflected genotypes of the chosen colonies consistent with either the HDR allele (founders 1650, 1651 and 1657) or deletions resulting from NHEJ (FIG. 5 panel a). Three of the DAZL piglets 203 were stillborn. Of the six piglets from APC modified cells, one was stillborn, three died within one week, and another died after 3 weeks, all for unknown reasons likely related to cloning. All six APC piglets were heterozygous for the intended HDR-edited allele and all but one either had an in-frame insertion or deletion of 3 bp on the second allele (FIG. 5a, b). Remaining animals are being raised for phenotypic analyses of spermatogenesis arrest (DAZL−/− founders) or development of colon cancer (APC+/−founders).

Template-driven introgression methods are detailed herein. Embodiments of the invention include template-driven introgression, e.g., by HDR templates, to place an APC or a DAZL allele into a non-human animal, or a cell of any species.

This method, and methods generally herein, refer to cells and animals. These may be chosen from the group consisting non-human vertebrates, non-human primates, cattle, horse, swine, sheep, chicken, avian, rabbit, goats, dog, cat, laboratory animal, and fish. The term livestock means domesticated animals that are raised as commodities for food or biological material. The term artiodactyl means a hoofed mammal of the order Artiodactyla, which includes cattle, deer, camels, hippopotamuses, sheep, and goats that have an even number of toes, usually two or sometimes four, on each foot.

### Gametogenesis and Gametogenic Promoters

Gametogenesis refers to the biological process by which germ line precursor cells undergo cell division and differentiation to form mature haploid gametes. Animals produce gametes through meiosis in the gonads. Primordial germ cells (PGCs) form gametogonia during development. Female gametognia undergo oogenesis, which has sub-processes of oocytogenesis, ootidogenesis, and maturation to form an ovum (sometimes referred to as oogenesis). Male gametognia undergo spermatogenesis. The gametogonia are precursors to male primary sperm cells (diploid) that undergo meiosis to produce spermatogonia) (diploid) that give rise to primary spermatocytes (diploid). Primary spermatocytes undergo meiosis to form secondary spermatocytes (haploid) that form spermatids (haploid) that develop into mature spermatozoa (haploid), also known as sperm cells. The seminiferous tubules of the testes are the starting point for the process, where stem cells adjacent to the inner tubule wall divide in a centripetal direction beginning at the walls and proceeding into the innermost part to produce spermatids. Maturation of the spermatids occurs in the epididymis. Research in mice or rats has shown that seminiferous tubules of a first animal can receive tissue and/or spermatogonial cells from a donor animal so that the donated cells mature into spermatozoa that functional. The recipient seminiferous tubules can effectively host the spermatogenic processes for donor cells.

Gametogenic promoters are promoters that are selective for gametogenic processes. Some gametogenic promoters act before the meiotic stages of gametogenesis while others are specifically activated at various points in the process of gametogenesis.

Embodiments include an exogenous gene placed into a cell or embryo under control of a promoter selective for gametogenesis or selectively activated during one or more gametogenic subprocesses chosen from the group consisting of oocytogenesis, ootidogenesis, oocyte maturation, spermatogenesis, maturation into spermatogonial cells, maturation into primary spermatocytes, maturation into secondary spermatocytes, maturation into spermatids, and maturation into sperm cells. Some promoters are generally active during gametogenesis while others are activated beginning at a certain subprocess but may continue through other phases of gametogenesis. Embodiments further include an exogenous gene placed into a cell or embryo under control of a tissue-specific promoter selective for gametogenic processes: for example, a tissue specific promoter selectively active in a tissue selected from the group consisting of testes, seminiferous tubules, and epididymis.

The cyclin A1 promoter is active not only in pachytene spermatocytes but also in earlier phases of spermatogenesis (Müller-Tidow et al., Int. J Mol. Med. 2003 March; 11(3):311-315; Successive increases in human cyclin A1 promoter activity during spermatogenesis in transgenic mice).

The promoter of SP-10 (−408/+28 or the −266/+28; referred to as SP-10 promoters) is directed only to spermatid-specific transcription. In fact, in transgenic mice, despite transgene integration adjacent to the pan-active CMV enhancer, the −408/+28 promoter maintained spermatid-specificity and no ectopic expression of the transgene resulted (P Reddi, et al. Spermatid-specific promoter of the SP-10 gene functions as an insulator in somatic cells. Developmental Biology 262(1):173-182, 2003). The 400-bp regulatory region of the stimulated by retinoic acid gene 8 (Stra8) promoter (referred to as the Stra8 promoter) is selectively active in meiotic and postmeiotic germ cells and not in undifferentiated germ cells (Antonangeli et al., Expression profile of a 400-bp Stra8 promoter region during spermatogenesis; Microscopy Research and Technique 72(11): 816-822, 2009).

The inventors have developed precise, high frequency editing of a variety of genes in about various livestock cells and/or animals that are useful for agriculture, for research tools, or for biomedical purposes. These livestock gene-editing processes include TALEN and CRISPR/Cas9 stimulated homology-directed repair (HDR) using, e.g., plasmid, rAAV and oligonucleotide templates. These processes have been developed by the inventors to achieve efficiencies that are so high that genetic changes can be made without reporters and/or without selection markers. Moreover, the processes can be used in the founder generation to make genetically modified animals that have only the intended change at the intended site. For instance, processes and data for targeting nucleases are provided in U.S. Ser. No. 14/154,906 filed Jan. 14, 2014, which is hereby incorporated herein by reference.

### Homology Directed Repair (HDR)

Homology directed repair (HDR) is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

### Targeted Nuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### Site-Specific Nuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. tracrRNA is another such tool. These are examples of targeted nuclease systems: these systems have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at or near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALs has been reported (PCT Application WO 2011/072246) wherein each DNA binding repeat is responsible for recognizing one base pair in the target DNA sequence. The residues may be assembled to target a DNA sequence. In brief, a target site for binding of a TALEN is determined and a fusion molecule comprising a nuclease and a series of RVDs that recognize the target site is created. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvCl, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a targeted endonuclease, a chimeric Zinc-Finger nuclease (ZFN) resulting from the fusion of engineered zinc-finger domains with the catalytic domain of a restriction enzyme such as Fold or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csm I, PI-See L PI-Tti L PI-Mtu I, I-Ceu I, I-See IL 1-See III, HO, PI-Civ I, PI-Ctr L PI-Aae I, PI-Bsu I, PI-Dha I, PI-Dra L PI-May L PI-Meh I, PI-Mfu L PI-Mfl I, PI-Mga L PI-Mgo I, PI-Min L PI-Mka L PI-Mle I, PI-Mma I, PI-30 Msh L PI-Msm I, PI-Mth I, PI-Mtu I, PI-Mxe I, PI-Npu I, PI-Pfu L PI-Rina I, PI-Spb I, PI-Ssp L PI-Fae L PI-Mja I, PI-Pho L PI-Tag L PI-Thy I, PI-Tko I, PI-Tsp I, I-MsoI.

A genetic modification made by TALENs or other tools may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting non-human vertebrates, non-human primates, cattle, horse, swine, sheep, chicken, avian, rabbit, goats, dog, cat, laboratory animal, and fish.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific Fold cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically modified animals are disclosed in, e.g., U.S. Pat. No. 8,106,255, U.S. 2012/0192298, U.S. 2011/0023159, and U.S. 2011/0281306.

### Vectors and Nucleic Acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

In general, type of promoter can be operably linked to a target nucleic acid sequence. Examples of promoters include, without limitation, tissue-specific promoters, constitutive promoters, inducible promoters, and promoters responsive or unresponsive to a particular stimulus. In some embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al., Hum. Gene Ther. 12:563, 2001 and Kiwaki et al. Hum. Gene Ther. 7:821, 1996.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. 89:6861, 1992, for a review of Cre/lox technology, and Brand and Dymecki, Dev. Cell 6:7, 2004. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be methylated using an SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinP1I endonuclease for 1 hour at 37° C. and assaying by agarose gel electrophoresis.

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. Nucleic Acids Res. 31:6873, 2003); Tol2 (Kawakami Genome Biology 8(Suppl.1):S7, 2007; Minos (Pavlopoulos et al. Genome Biology 8(Suppl.1):S2, 2007); Hsmar1 (Miskey et al.) Mol Cell Biol. 27:4589, 2007); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### Genetically Modified Animals

Animals may be modified using TALENs or other genetic engineering tools, including recombinase fusion proteins, or various vectors that are known. A genetic modification made by such tools may comprise disruption of a gene. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying animals are further detailed in U.S. Ser. No. 13/404,662 filed Feb. 24, 2012, Ser. No. 13/467,588 filed May 9, 2012, and Ser. No. 12/622,886 filed Nov. 10, 2009 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Embodiments of trans-acting gene are, e.g., genes that encode targeting endonucleases. Inactivation of a. gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knockout animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al., Proc. Natl. Acad. Sci. USA 82:6148-1652, 1985), gene targeting into embryonic stem cells (Thompson et al., Cell 56:313-321, 1989), electroporation of embryos (Lo, Mol. Cell. Biol. 3:1803-1814, 1983), sperm-mediated gene transfer (Lavitrano et al., Proc. Natl. Acad. Sci. USA 99:14230-14235, 2002; Lavitrano et al. Reprod. Fert. Develop. 18:19-23, 2006), and in vitro transformation of somatic cells, such as cumulus or mammary cells, or adult, fetal, or embryonic stem cells, followed by nuclear transplantation (Wilmut et al., Nature 385:810-813, 1997; and Wakayama et al., Nature 394:369-374, 1998). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygousity would normally be required. If a particular gene is inactivated by an RNA interference or dominant negative strategy, then heterozygosity is often adequate.

Typically, in pronuclear microinjection, a nucleic acid construct is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO2. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×105 sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO2 atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs can be injected into one of the pronuclei. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded. Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al., Science 280:1256-1258, 1998 and U.S. Pat. No. 6,548,741. For pigs, recipient females can be checked for pregnancy approximately 20-21 days after transfer of the embryos.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animal have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., 1989, Molecular Cloning, A Laboratory Manual, second edition, Cold Spring Harbor Press, Plainview; NY. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis Genetic Engineering News 12:1, 1992; Guatelli et al., Proc. Natl. Acad. Sci. USA 87:1874, 1990; and Weiss Science 254:1292, 1991. At the blastocyst stage, embryos can be individually processed for analysis by PCR, Southern hybridization and splinkerette PCR (see, e.g., Dupuy et al. Proc. Natl. Acad. Sci. USA 99:4495, 2002).

Expression of a nucleic acid sequence encoding a polypeptide in the tissues of transgenic pigs can be assessed using techniques that include, for example, Northern blot analysis of tissue samples obtained from the animal, in situ hybridization analysis, Westem analysis, immunoassays such as enzyme-linked inununosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically modified animal comprises inducing RNA interference against a target gene and/or nucleic acid such that expression of the target gene and/or nucleic acid is reduced.

For example the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al., Nature 391:806, 1998; Romano and Masino Mol. Microbiol. 6:3343, 1992; Cogoni et al., EMBO J. 15:3153, 1996; Cogoni and Masino Nature 399:166, 1999; Misquitta and Paterson Proc. Natl. Acad. Sci. USA 96:1451, 1999; and Kennerdell and Carthew Cell 95:1017, 1998. Constructs for shRNA can be produced as described by McIntyre and Fanning BMC Biotechnology 6:1, 2006. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically modified animal such as a livestock animal that express an RNAi directed against a gene, e.g., a gene selective for a developmental stage. The RNAi may be, for instance, selected from the group consisting of siRNA, shRNA, dsRNA, RISC and miRNA.

### Inducible Systems

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements. An example of an inducible system is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a foxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically modified animal such as a livestock animal that comprise a gene under control of an inducible system. The genetic modification of an animal may be genomic or mosaic. The inducible system may be, for instance, selected from the group consisting of Tet-On, Tet-Off, Cre-lox, and Hif1alpha. An embodiment is a gene set forth herein, e.g., in the group consisting of DAZL, vasa, CatSper, KCNU1, DNAH8, and Testis expressed gene 11 (Text 1).

### Dominant Negatives

Genes may thus be disrupted not only by removal or RNAi suppression but also by creation/expression of a dominant negative variant of a protein which has inhibitory effects on the normal function of that gene product. The expression of a dominant negative (DN) gene can result in an altered phenotype, exerted by a) a titration effect; the DN PASSIVELY competes with an endogenous gene product for either a cooperative factor or the normal target of the endogenous gene without elaborating the same activity, b) a poison pill (or monkey wrench) effect wherein the dominant negative gene product ACTIVELY interferes with a process required for normal gene function, c) a feedback effect, wherein the DN ACTIVELY stimulates a negative regulator of the gene function.

### Founder Animals, Animal Lines, Traits, and Reproduction

Founder animals may be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that all of the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Science, 40:123-137, 1994, U.S. Pat. No. 7,709,206, U.S. 2001/0016315, U.S. 2011/0023140, and U.S. 2005/0153317. An animal line may include a trait chosen from a trait in the group consisting of a production trait, a type trait, a workability trait, a fertility trait, a mothering trait, and a disease resistance trait. Further traits include expression of a recombinant gene product.

Animals with a desired trait or traits may be modified to prevent their reproduction. Animals that have been bred or modified to have one or more traits can thus be provided to recipients with a reduced risk that the recipients will breed the animals and misappropriate the value of the traits to themselves.

Breeding of animals that require administration of a compound to induce fertility or sexual fertility may advantageously be accomplished at a treatment facility. The treatment facility can implement standardized protocols on well-controlled stock to efficiently produce consistent animals. The animal progeny may be distributed to a plurality of locations to be raised. Farms and farmers (a term including a ranch and ranchers) may thus order a desired number of progeny with a specified range of ages and/or weights and/or traits and have them delivered at a desired time and/or location. The recipients, e.g., farmers, may then raise the animals and deliver them to market as they desire.

Embodiments include delivering (e.g., to one or more locations, to a plurality of farms) a genetically modified livestock animal having a gene disrupted so that the animal is incapable of sexual reproduction. The animal may have one or more traits (for example one that expresses a desired trait or a high-value trait or a novel trait or a recombinant trait). Embodiments further include providing said animal and/or breeding said animal.

### Recombinases

Embodiments of the invention include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The disclosure of U.S. Pub. 2011/0059160 (U.S. Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae. Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

### Compositions and Kits

The present invention also provides compositions and kits containing, for example, nucleic acid molecules encoding site-specific endonucleases, CRISPR, Cas9, ZNFs, TALENs, RecA-gal4 fusions, polypeptides of the same, compositions containing such nucleic acid molecules or polypeptides, or engineered cell lines. An HDR may also be provided that is effective for introgression of an indicated allele. Such items can be used, for example, as research tools, or therapeutically.

### EXAMPLES

Materials and methods, including making of TALENs, are generally as described in U.S. Ser. No. 13/594,694 filed Aug. 24, 2012, unless otherwise indicated.

### Example 1

### TALENs for Y-Chromosome Modification

Transfection—Fibroblasts are cultured and transfected by nucleofection as previously described. (Carlson et al., 2011) Transposon components total 1 μg in the

Experiments. For Homology-Dependent Repair (HDR) analysis, the best performing condition for Double-Strand-Break (DSB) induction are chosen and repair template is added at equal, 3 and 10 fold excess to TALEN plasmid. Cell culture—Isolation of individual colonies is conducted by selection in 96-well plates at pre-determined densities to result in colonies in 30-50% of wells. Indel detection populations—Primers flanking the target sites are designed to result in amplicons ˜500 bp. PCR amplicons are treated with SURVEYOR® Nuclease (Transgenomic, Omaha Nebr.) as suggested, and resolved on 8-10% polyacrylamide gels. A portion of amplicons from indel positive blastocysts are cloned and sequenced to characterize the mutation. Indel detection colonies—Primers flanking the target site as used above are used for amplification using the High Resolution Melt analysis qPCR master mix (Invitrogen) and melting curves analysis will be conducted. The variation in melt profile of the real time PCR product will distinguish clones carrying TALEN induced mutation from wild type sequence. Normal variation in the melting temperature of amplicons derived non-transfected control cells will be used as a reference. Amplicons with melt profiles outside of the normal variation are cloned and sequenced to characterize mutations. Y-Targeting detection—PCR assays are developed with a primer outside of the homology arms and one within to result in a product only possible if homologous recombination has occurred. PCR-positive colonies are validated by Whole Genome Amplification Southern blotting. WGA Southern Blotting to confirm Y-targeting-WGA is performed on individual clones using half reactions of the REPLI-g Mini Kit (Qiagen, Valencia, Calif.) according to the “Amplification of Blood or Cells” protocol. Probes for Southern Blotting are hybridized to validate 5′ and 3′ junctions of targeted cells. FACS-Fresh semen is prepared for sorting of X- and Y-bearing sperm cells by placing 15 million spermatozoa in 1 ml of BTS with Hoechst 33342 added to a concentration of 6.25 uM. This preparation is incubated for 45 min at 35° C. X- and Y-bearing sperm are sorted by DNA content using a modified flow cytometer with standard modifications for sperm sorting. (Johnson et al., 1987; Johnson and Pinkel, 1986) Accuracy of sorted populations is determined by quantitative PCR for X and Y targets. Serum hormone measurements—Blood serum levels of testosterone and FSH are evaluated using commercially ELISA kits from Endocrine Technologies (Newark, Calif.).

Four TALEN pairs were made that are directed against two candidate loci for Y chromosome gene addition (FIG. 4). The first candidate is located 1.7 kb 3′ of SRY, beyond the two highest ranking poly-adenylation signals. A second candidate locus is the Y-specific intron of the AMELY gene. These loci are predicted to lie outside of the pseudoautosomal boundary of SSCY based on comparison with cattle and pig:cattle comparative gene mapping data. (Quilter et al., 2002; Van Laere et al., 2008) As such, they are not capable of undergoing recombination with SSCX or autosomes and thus expected to be maintained on SSCY across numerous generations. Three of four TALENs pairs tested revealed high activity (FIG. 4).

### Example 2

### Isolation of Mono- and Bi-Allelic KO Clones

Carlson et al. 2012 described modification of target genes in livestock wherein transgenic primary fibroblasts were effectively expanded and isolated as colonies when plated with non-transgenic fibroblasts (feeder-cells) at standard densities (>150 cells/cm2) and subjected to drug selection using the transposon co-selection technique applied above (Carlson et al., Transgenic Res. 20:1125, 2011 and U.S. Pub 2012/0220037 filed May 9, 2012). These techniques are useful for making genetic changes to somatic cells that can be used to clone animals.

As an example, puromycin resistant colonies for cells treated with six TALEN pairs were isolated and their genotypes evaluated. In general, the proportion of indel positive clones was similar to predictions made based on day 3 modification levels. Bi-alleic knockout clones were identified for 6 of 7 different TALEN pairs, occurring in up to 35 percent of indel positive cells. In the majority of examples, indels were homozygous (same indel on each allele) rather than unique indels on each allele suggesting that sister chromatid-templated repair is common. Notably, among modified clones, the frequency of bi-alleic modification (17-60%) for the majority of TALEN pairs exceed predictions based on day 3 modification levels (10-17%) if chromosome cleavages are treated as independent events.

### Example 3

### TALEN Mediated DNA Cleavage as a Target for HDR in Livestock Cells

A TALEN pair (LDLR4.2) targeted to the fourth exon of the swine low density lipoprotein receptor (LDLR) gene was co-transfected with the supercoiled plasmid Ldlr-E4N-stop, which contains homology arms corresponding to the swine LDLR gene and a gene-trap enabling expression of Neomycin phosphotransferase upon HDR. After 3 days of culture PCR analysis revealed that, even without antibiotic selection, a band corresponding to an HDR event could be detected at the targeted locus at 30° C. Selection of populations of cultured cells for 14 days with geneticin (G418) resulted in significant enrichment of HDR cells.

### Example 4

### Single Stranded DNA for Templating

Tan et al. 2013 described use of single stranded DNA of template-driven modification of genes. Single stranded oligodeoxynucleotides (ssODNs) are an effective template for TALEN stimulated HR. Loci were targeted to introgress the 11 base pair Belgian Blue cattle mutation into Wagyu cells. Two 76 base pair ssODNs were designed to mimic either the sense or antisense strand of the BB GDF8 gene including the 11 base pair deletion. Four micrograms of TALEN encoding plasmids were transfected into Wagyu cells, and 0.3 nMol of ssODNs were either co-transfected with TALENS (N) or delivered 24 hours after TALEN nucleofection by either MirusLT1 (M) reagent or Lipofectamine LTX reagent (L). Semi-quantitative PCR at day three indicated an allele conversion frequency of up to 5% in conditions where ssODNs were delivered with LIPOFECTAMINE LTX reagent 24 hours after TALEN transfection. No difference in PCR signal was observed between sense and antisense ssODNs designed against the target.

### Example 5

### Alleles Introduced into Pig (Ossabaw) Cells Using Oligo HDR

Tan et al. (2013) describe modifying cells with a combination of mRNA encoded TALENs and single-stranded oligonucleotides to place an allele that occurs naturally in one species to another species (interspecific migration). Piedmontese GFD8 SNP C313Y, were chosen as an example and was introduced into Ossabow swine cells. No markers were used in these cells at any stage. A similar peak in HDR was observed between pig and cattle cells at 0.4 nmol ssODN, (not shown) however, HDR was not extinguished by higher concentrations of ssODN in Ossabaw fibroblasts.

### Example 6

### CRISPR/Cas9 Design and Production

Gene specific gRNA sequences were cloned into the Church lab gRNA vector (Addgene ID: 41824) according their methods. The Cas9 nuclease was provided either by co-transfection of the hCas9 plasmid (Addgene ID: 41815) or mRNA synthesized from RCIScript-hCas9. This RCIScript-hCas9 was constructed by sub-cloning the XbaI-AgeI fragment from the hCas9 plasmid (encompassing the hCas9 cDNA) into the RCIScript plasmid. Synthesis of mRNA was conducted as above except that linearization was performed using KpnI.

### Example 7

### CRISPR/Cas9

CRISPR/Cas9 mediated HDR was used to introgress the p65 S531P mutation from warthogs into conventional swine. Referring to FIG. 5, at Panel a) The S531P missense mutation is caused by a T-C transition at nucleotide 1591 of porcine p65 (RELA). The S-P HDR template includes the causative TC transition mutation (oversized text) which introduces a novel XmaI site and enables RFLP screening. Two gRNA sequences (P65_G1S and P65_G2A) are shown along with the p65.8 TALENs used in previous experiments. At panel b) Landrace fibroblasts were transfected with S-P-HDR oligos (2 μM), two quantities of plasmid encoding hCas9 (0.5 μg v.s. 2.0 μg); and five quantities of the G2A transcription plasmid (0.05 to 1.0 μg). Cells from each transfection were split 60:40 for culture at 30 and 37° C. respectively for 3 days before prolonged culture at 37° C. until day 10. Surveyor assay revealed activity ranging from 16-30%. Panel's c and d) RFLP analysis of cells sampled at days 3 and 10. Expected cleavage products of 191 and 118 bp are indicated by black arrows. Despite close proximity of the DSB to the target SNP, CRISPR/Cas9 mediated HDR was less efficient than TALENs for introgression of S531P. Individual colonies were also analyzed using each gRNA sequence.

### Example 8

### CRISPR/Cas9

Comparison of TALENs and CRISPR/Cas9 mediated HDR at porcine APC. Referring to FIG. 6, at panel a) APC14.2 TALENs and the gRNA sequence APC14.2 G1a are shown relative to the wild type APC sequence. Below, the HDR oligo is shown which delivers a 4 bp insertion (see text) resulting in a novel HindIII site. Pig fibroblasts transfected with 2 μM of oligo HDR template, and either 1 μg TALEN mRNA, 1 μg each plasmid DNA encoding hCas9 and the gRNA expression plasmid; or 1 μg mRNA encoding hCas9 and 0.5 μg of gRNA expression plasmid, were then split and cultured at either 30 or 37° C. for 3 days before expansion at 37° C. until day 10. Panel b) Charts displaying RFLP and Surveyor assay results. As previously determined TALEN stimulated HDR was most efficient at 30° C., while CRISPR/Cas9 mediated HDR was most effective at 37° C. For this locus, TALENs were more effective than the CRISPR/Cas9 system for stimulation of HDR despite similar DNA cutting frequency measured by SURVEYOR assay. In contrast to TALENs, there was little difference in HDR when hCas9 was delivered as mRNA versus plasmid.

### Example 9

### Targeting the Y-Chromosome

A combination of TALENs and plasmid homology cassettes were used to target the mCaggs-EGFP cassette to the Y-chromosome. For the purposes of this experiment, the positive orientation is when both the transgene cassette and the endogenous gene (SRY or AMELY) are in the same orientation, the negative orientation is when they are in opposite orientation. One microgram of TALEN mRNA plus 500 ng of the homology cassette was mixed with 600,000 cells in a single 100 ul electroporation. Cells were transfected using the NEON electroporation system (Life Technologies), cultured for 3 days at 30° C., and plated at low density for derivation of single cell derived colonies. Colonies were analyzed for correct targeting of the Y chromosome by junction PCR using one primer outside of the homology arms and a second primer within the transgene cassette. Several colonies were positive for the expected amplicon. FIG. 8 is a summary of the results shown in FIG. 7. Clones positive for Y-targeting ranged from 6-24 percent. The orientation of the transgene cassette appeared to have some effect on the efficiency of Y-targeting.

### Example 10

### Short Homology Targeting of the Y Chromosome

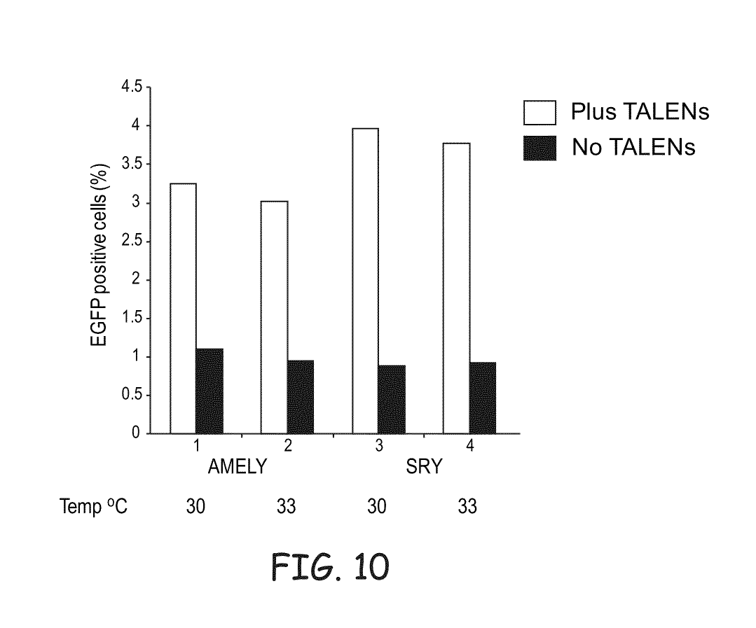

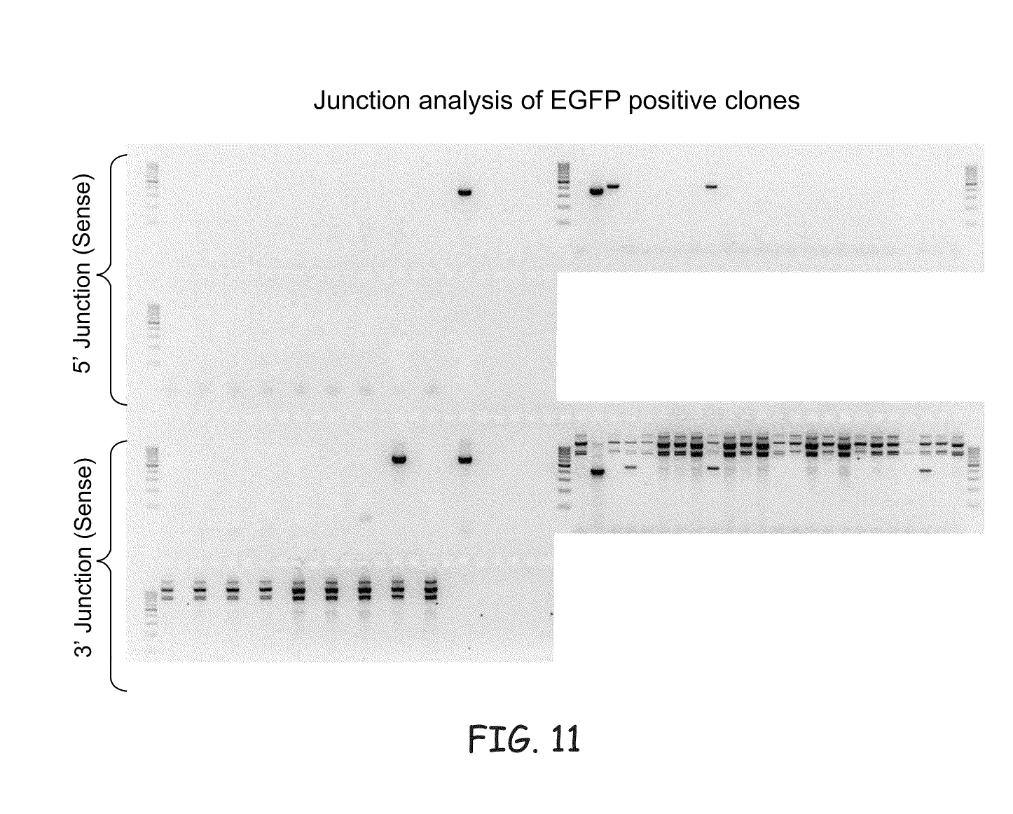

As an alternative to plasmid homology cassettes, linear templates with short (50-100 bp) homology arms were developed to target AMELY and SRY sites. The homology templates were created by PCR amplification of the ubiquitin EGFP cassette using primers that bound to the 5′ and 3′ end of the cassette and included a tail corresponding to the sequence 5′ and 3′ of the presumptive TALEN induced double strand break as described in Orlando et al. 2010 (NAR; 2010 August; 38(15)). The primers included phosphorthioate linkages between the first two nucleotides to inhibit degradation by endogenous nucleases. Two micrograms of TALEN mRNA (or none as control) and 1 ug of short homology template specific to each site was included in a typical 100 ul electroporation. After electroporation, the cells were divided for culture at either 30 or 33° C. for three days, followed by junction PCR to test for Y-targeting. Cells cultured at 30 or 33° C. were positive for Y-targeting at both the 5′ and 3′ junction, though product intensity suggests Y-targeting is more efficient at 30° C. For each site, amplicons corresponding to correct Y-targeting was dependent on TALENs, note the top band of the SRY 3′ junction is non-specific background signal. Cell populations cultured for 14 days post-transfection should no longer express non-integrated templates. FACs for EGFP was conducted on day 14 populations to determine if the combination of TALENs plus the short homology template, versus template alone, increases the proportion of EGFP positive cells. Indeed, EGFP positive cells were ˜3-fold enriched when TALENs were included and little temperature effect was observed (FIG. 10). Individual EGFP positive colonies were genotyped for Y-targeting. For AMELY, 0/5 (0%) and 2/5 (20%) of EGFP positive colonies were also positive for Y-targeting from cells initially cultured at 30 or 33° C. respectively (FIG. 11). For SRY, 5/24 (21%) and 0/9 (0%) of EGFP positive colonies were also positive for Y-targeting from cells initially cultured at 30 or 33° C. respectively (FIG. 11).

### Example 11

### TALEN HDR for Gene Knockout in Pigs

To generate pigs with custom designed knockout allele, we treated cells with TALENs and oligos as described in Tan et al., 2013. For this set of experiments, TALENs and oligo templates were designed to target swine DAZL or APC respectively, followed by isolation of single colonies and screening for the novel restriction site introduced by oligo HDR FIG. 12 is a montage of experimental results showing cloned pigs with HDR alleles of DAZL and APC. Panel a) is a restriction fragment length polymorphism (RFLP) analysis of cloned piglets derived from DAZL- and APC-modified landrace and Ossabaw fibroblasts, respectively. Expected RFLP products for DAZL founders are 312, 242, and 70 bp (open triangles), and those for APC are 310, 221, and 89 bp (filled triangles). The difference in size of the 312-bp band between WT and DAZL founders reflects the expected deletion alleles. Panel b) Sequence analysis confirming the presence of the HDR allele in three of eight DAZL founders, and in six of six APC founders. Blocking mutations in the donor templates (HDR) are in boxes, and inserted bases are underlined. The bold text in the top WT sequence indicates the TALEN-binding sites. Panel c) Photographs of DAZL (Left) and APC (Right) founder animals.

### Example 12

### DAZL-KO Boars Lack Germ Cells

FIG. 13 is a microphotographic montage showing that DAZL KO pigs show a lack of spermatogenesis and a complete absence of germ cells. a. H&E staining of DAZL KO seminiferous tubules from the inner portion of the testes shows a complete absence of spermatogonia. b. H&E staining of DAZL KO seminiferous tubules from the outer portion of the testes also shows a complete absence of spermatogonia. c. Ubiquitin carboxy-terminal hydrolase L1 (UCH-LI), a marker of spermatogonia is present in wild type pig testes. d. UCH-LI is absent in DAZL KO testes, indicating an absence of spermatogonia. e. Actelyated a-tubulin is present in the seminiferous tubules of wild type pig testes, indicating the presence of spermatogonia. f. DAZL KO pig seminiferous tubules are negative for acetylated a-tubulin demonstrating a lack of germ cells in these animals.

All publications, patent applications, and patents set forth herein are hereby incorporated herein by reference for all purposes; in case of conflict, the instant specification controls.

### FURTHER DESCRIPTION

Embodiments include, for instance, all of the following, which are numbered for reference. 1: A genetically modified animal, the animal comprising a genetic modification to disrupt a target gene selectively involved in gametogenesis, wherein: the disruption of the target gene prevents formation of functional gametes of the animal. 2: The animal of 1 wherein the disruption of the gene comprises an insertion, deletion, or substitution of one or more bases in a sequence encoding the target gene and/or a cis-regulatory element thereof. 3: The animal of 1 wherein the disrupted gene is disrupted by: removal of at least a portion of the gene from a genome of the animal, alteration of the gene to prevent expression of a functional factor encoded by the gene, or a trans-acting factor. 4: The animal of 3 wherein the target gene is disrupted by the trans-acting factor, said trans-acting factor being chosen from the group consisting of interfering RNA and a dominant negative factor, with said trans-acting factor being expressed by an exogenous gene or an endogenous gene. The trans acting factor can be, e.g., a targeted nuclease. 5: The animal of 1 wherein the disruption of the target gene is under control of an inducible system. 6: The animal of 5 wherein the inducible system comprises a member of the group consisting of Tet-On, Tet-Off, Cre-lox, Hif1alpha, RHEOSWITCH, ecdysone gene switch, and cumate gene switch. 7: The animal of 1 wherein the target gene is chosen from the group consisting of DAZL, vasa, CatSper, KCNU1, DNAH8, and Testis expressed gene 11 (Tex11). 8: The animal of 1 wherein the target gene is on an X chromosome or an autosome. 9: The animal of 1 wherein the target gene is on a Y chromosome. 10: The animal of 1 wherein the disruption of the target gene selectively inhibits formation of a male gamete or a female gamete. 11: The animal of 1 wherein the target gene is chosen from the group consisting of TENR, ADAM1a, ADAM2, ADAM, alpha4, ATP2B4 gene, a CatSper gene subunit, CatSper1, CatSper2, CatSper3, Catsper4, CatSperbeta, CatSpergamma, CatSperdelta, Clamegin, Complexin-I, Sertoli cell androgen receptor, Gasz, Ra175, Cib1, Cnot7, Zmynd15, CKs2, and Smcp. 12: The animal of 1 wherein the target gene is necessary for spermatogenesis, wherein disruption of the gene selectively inhibits spermatogenesis. 13: The animal of 12 wherein the target gene comprises Testis expressed gene 11 (Tex11). 14: The animal of 1 wherein the target gene is necessary for sperm motility, sperm acrosome fusion, or sperm syngamy, wherein disruption of the target gene selectively inhibits one or more of sperm motility, sperm acrosome fusion, or sperm syngamy. 15: The animal of 14 wherein disruption of the target gene selectively inhibits sperm motility and the gene is chosen from the group consisting of TENR, ADAM1a, ADAM3, Atp1a4, and ATP2B4. 16: The animal of 14 wherein disruption of the target gene selectively inhibits sperm acrosome fusion and the gene is chosen from the group consisting of ADAM2, ADAM3, CatSper, Clamegin, and Complexin-I. 17: The animal of 1 wherein the animal is chosen from the group consisting of non-human vertebrates, non-human primates, cattle, horse, swine, sheep, chicken, avian, rabbit, goats, dog, cat, laboratory animal, and fish. 18: The animal of 1 being sterile, male, and unable to produce functional sperm. 19: The animal of 18 wherein the target gene comprises DAZL. 20: The animal of 1 being a recipient of donor cells that give rise to functional donor sperm having a haploid donor chromosomal complement of the donor. 21: The animal of 20 wherein the donor cells further comprise a gene for expressing a transgenic recombinant protein. 22: The animal of 1 comprising a transgenic trait chosen from the group consisting of a production trait, a type trait, a workability trait, a fertility trait, a mothering trait, and a disease resistance trait.

23: A process of preparing cells of an animal comprising introducing, into an organism chosen from the group consisting of a cell and an embryo, an agent that specifically binds to a chromosomal target site of the cell and causes a double-stranded DNA break to disrupt a gene to selectively disrupt gametogenesis, with the agent being chosen from the group consisting of a targeted endonuclease, an RNA, and a recombinase fusion protein. 24: The process of 23 wherein the agent is the targeted endonuclease and comprises a TALEN or a TALEN pair that comprises a sequence to specifically bind the chromosomal target site, and creates the double stranded break in the gene or creates the double stranded break in the chromosome in combination with a further TALEN that creates a second double stranded break with at least a portion of the gene being disposed between the first break and the second break. 25: The process of 23 wherein the agent comprises the targeting nuclease and is selected from the group consisting of zinc finger nucleases, meganucleases, RNA-guided nucleases, or CRISPR/Cas9. 26: The process of 24 further comprising co-introducing a recombinase into the organism with the targeted endonuclease. 27: The process of 23 wherein the introducing the agent into an organism comprises a method chosen from the group consisting of direct injection of the agent as peptides, injection of mRNA encoding the agent, exposing the organism to a vector encoding the agent, and introducing a plasmid encoding the agent into the organism. 28: The process of 23 wherein the agent is the recombinase fusion protein, with the process comprising introducing a targeting nucleic acid sequence with the fusion protein, with the targeting nucleic acid sequence forming a filament with the recombinase for specific binding to the chromosomal site. 29: The process of 23 wherein the recombinase fusion protein comprises a recombinase and Gal4. 30: The process of 23 further comprising introducing a nucleic acid into the organism, wherein the nucleic acid is introduced into the genome of the organism at a site of the double-stranded break or between the first break and second break. For instance, homology dependent repair (HDR) can be a mechanism for the introduction, e.g., with an oligo-based HDR. 31: The process of 23 wherein the cell is chosen from the group consisting of an in vitro cell, an in vitro primary cell, a zygote, an oocyte, a gametogenic cell, a sperm cell, an oocyte, a stem cell, and a zygote. 32: The process of 31 wherein the cell is a zygote or embryo, and comprising implanting the zygote in a surrogate mother. 33: The process of 31 comprising cloning the cell. 34: The process of 33 wherein cloning the cell is performed with a process chosen from the group consisting of somatic cell nuclear transfer and chromatin transfer. 35: The process of 23 further comprising introducing a nucleic acid template into the cell, with the template having ends that are substantially homologous to ends produced by the break. Further, the template may guide HDR. 36: The process of 23 wherein the agent is introduced as a nucleic acid that is transcribed by the cell to make the agent. 37: The process of 23 wherein the animal is chosen from the group consisting of non-human vertebrates, non-human primates, cattle, horse, swine, sheep, chicken, avian, rabbit, goats, dog, cat, laboratory animal, and fish. 38: The process of 23 wherein the disruption of the gene is under control of an inducible system. 39: The process of 23 wherein the disrupted gene is chosen from the group consisting of DAZL, vasa, CatSper, KCNU1, DNAH8, PIWIL4 (MIWI2), PIWIL2 (MIWI) and Testis expressed gene 11 (Tex11).

40: An in vitro cell comprising an agent that specifically binds to a chromosomal target site of the cell and causes a double-stranded DNA break to disrupt a gene to selectively disrupt gametogenesis, with the agent being chosen from the group consisting of a targeting endonuclease, and a recombinase fusion protein. 41: The cell of 40 wherein the agent is a TALEN or a TALEN pair that comprises a sequence to specifically bind the chromosomal target site, and creates the double stranded break in the gene or creates the double stranded break in the chromosome in combination with a further TALEN that creates a second double stranded break with at least a portion of the gene being disposed between the first break and the second break. Also, the cell of 40 wherein the agent comprises the targeted nuclease and is selected from the group consisting of zinc finger nucleases, Tal-effector nucleases, RNA-guided nucleases (eg. CRISPR/Cas9), meganucleases. 42: The cell of 41 wherein the chromosome is a Y chromosome.

43: A genetically modified animal comprising a genomic modification to a Y chromosome, with the modification comprising an insertion, a deletion, or a substitution of one or more bases of the chromosome. For instance wherein the animal is chosen from the group consisting of non-human vertebrates, non-human primates, cattle, horse, swine, sheep, chicken, avian, rabbit, goats, dog, cat, laboratory animal, and fish. 44: The animal of 43 wherein the modification is made at a gene of the Y chromosome. 45: The animal of 43 wherein the modification comprises an insertion of an exogenous nucleic acid encoding a factor that disables a gamete that comprises the Y chromosome. 46: The animal of 43 wherein the exogenous nucleic acid expresses a factor chosen from the group consisting of an interfering RNA, a targeted nuclease, and a dominant negative. 47: The animal of 43 wherein the exogenous nucleic acid expresses a factor chosen from the group consisting of an apoptotic factor and an endonuclease. 48: The livestock animal of 43 wherein expression of the exogenous nucleic acid is under control of an inducible system.

49: A genetically modified animal, the animal comprising an exogenous gene on a chromosome, the gene being under control of a gene expression element that is selectively activated in gametogenesis. For instance wherein the animal is chosen from the group consisting of non-human vertebrates, non-human primates, cattle, horse, swine, sheep, chicken, avian, rabbit, goats, dog, cat, laboratory animal, and fish. 50: The animal of 49 wherein the chromosome is a Y chromosome. 51: The animal of 49 wherein the exogenous gene comprises encoding for a nuclease. Also, the animal of 50 wherein the nuclease is a targeted endonuclease. 52: Also, the animal wherein the targeted nuclease is chosen from the group consisting of TALENs, Zinc finger nucleases, meganucleases, or CRISPR/Cas9. Also, wherein the targeted endonuclease specifically binds to, and cleaves, a target gene. 53: The animal of 51 wherein the target gene is a member of the group consisting of DAZL, vasa, CatSper, KCNU1, DNAH8, PIWIL4, PIWIL2, and Testis expressed gene 11 (Tex11). 54: The animal of 49 wherein the gene expression element comprises a promoter, e.g., a cyclin A1 promoter, or a gene expression element. MicroRNA sites may be used. 55: The animal of 49 wherein the gene expression element is selective for spermatogenesis and is chosen from the group consisting of an SP-10 promoter, a Stra8 promoter, C-Kit, ACE, and protamine. 56: The animal of 49 wherein the gene expression element is selective for oogenesis and is chosen from the group consisting of a Nobox, Oct4, Bmp15, Gdf9, Oogenesin1 and Oogenesin2. 57: The animal of 49 wherein the exogenous gene inactivates a gene selectively required for production of a male progeny, and sexual reproduction of the animal produces only female progeny. 58: The animal of 49 wherein the exogenous gene inactivates a gene selectively required for production of a female progeny, and sexual reproduction of the animal produces only male progeny. 59: The animal of 49 wherein the exogenous gene expresses a factor that is fatal to a cell to thereby destroy only male or female gametes. 60: The animal of 59 wherein the factor comprises an apoptotic factor or toxic gene product. 61: The animal of 59 wherein the factor is apoptotic and the exogenous gene is chosen from the group consisting of FAS, BAX, CASP3, and SPATA17. 62: The animal of 59 wherein the factor is toxic and the gene is chosen from the group consisting of TOXIN gene, Barnase, diphtheria toxin A, thymidine kinase, and ricin toxin. 63: The animal of 59 wherein the factor comprises an endonuclease. 64: The animal of 63 wherein the (endo)nuclease is a broad spectrum nuclease for general degradation of RNA and/or DNA, or otherwise useful to disrupt general cell activity, e.g., DICER. 65: The animal of 49 being a male or female that is genetically sterile, with the exogenous gene expressing a factor that interferes with a second gene that is selective for spermatogenesis or oogenesis, respectively, thereby preventing successful sexual reproduction by the animal. 66: The animal of 65 wherein the factor is chosen from the group consisting of a targeting endonuclease, e.g., TALENs, an interfering RNA, and a dominant negative. 67: The animal of 65 wherein interference with the second gene selectively inhibits sperm motility, sperm acrosome fusion, or sperm syngamy and/or the animal of 65 wherein the exogenous gene comprises sperm dynein interfering protein (SDIP).

68: A genetically modified animal comprising a genetically infertile male livestock animal that generates functional donor spermatozoa without production of functional native spermatozoa. For instance wherein the animal is chosen from the group consisting of non-human vertebrates, non-human primates, cattle, horse, swine, sheep, chicken, avian, rabbit, goats, dog, cat, laboratory animal, and fish. 69: The animal of 68 wherein the animal sexually reproduces progeny of the donor. 70: The animal of 68 wherein a genome of the donor further comprises a trait or chosen from the group consisting of a production trait, a type trait, a workability trait, a fertility trait, a mothering trait, and a disease resistance trait. 71: A herd comprising a plurality of the animals of 68. 72: The herd of 71 wherein the donor spermatids of the animals carry genotypically identical chromosomes (alternatively: carry the same germplasm).

73: A genetically modified animal, the animal comprising an exogenous gene on a chromosome, the gene expressing a factor that controls a gender of progeny of the animal, with said animal producing progeny of only one gender. For instance wherein the animal is chosen from the group consisting of non-human vertebrates, non-human primates, cattle, horse, swine, sheep, chicken, avian, rabbit, goats, dog, cat, laboratory animal, and fish. 74: The animal of 73 wherein the chromosome is a Y chromosome. 75: The animal of 74 wherein the exogenous gene expresses a factor that is fatal to a cell to thereby destroy only male or female gametes or embryos. 76: The animal of 75 wherein the exogenous gene comprises encoding for a nuclease. 77: The animal of 76 wherein the nuclease is a broad spectrum nuclease for general degradation of RNA and/or DNA, or otherwise useful to disrupt general cell activity, e.g., DICER. 78: The animal of 76 wherein the nuclease is a targeting endonuclease. 79: The animal of 75 wherein the factor comprises an apoptotic factor or toxic gene product. 80: The animal of 77 wherein the factor is apoptotic and the exogenous gene is chosen from the group consisting of FAS, BAX, CASP3, and SPATA17. 81: The animal of 79 wherein the factor is toxic and the gene is chosen from the group consisting of TOXIN gene, Barnase, diphtheria toxin A, thymidine kinase, and ricin toxin. 82: The animal of 75 wherein the exogenous gene encodes a fusion of the factor and a microRNA. 83: The animal of 73 wherein the factor comprises a targeted nuclease that specifically binds to, and cleaves, a target sequence of a chromosome. 84: The animal of 83 wherein the targeted endonuclease is chosen from the group consisting of TALENs, Zinc finger nucleases, guided RNA targeting nucleases, RecA-fusion proteins, and meganucleases. 85: The animal of 73 wherein the factor is chosen from the group consisting of a targeting endonuclease, e.g, TALENs, an interfering RNA, and a dominant negative. 86: The animal of 83 wherein the exogenous gene inactivates a gene selectively required for production of a male progeny, and sexual reproduction of the animal produces only female progeny. For instance, SRY or a gene for MIS (Mullerian inhibiting substance) may be disrupted.

1. A genetically modified livestock animal, the animal comprising a genetic modification to disrupt a target gene selectively involved in gametogenesis, wherein the disruption of the target gene prevents formation of functional gametes of the animal.

2. The livestock animal of claim 1 wherein the disruption of the target gene is under control of an inducible system.

3. The animal of claim 1 wherein the target gene is chosen from the group consisting of DAZL, vasa, CatSper, KCNU1, DNAH8, and Testis expressed gene 11 (Tex11).

4. The animal of claim 1 wherein the target gene is on an X chromosome, Y chromosome, or an autosome.

5. The animal of claim 1 wherein the disruption of the target gene selectively inhibits formation of a male gamete or a female gamete.

6. The animal of claim 1 wherein the target gene is chosen from the group consisting of TENR, ADAM1a, ADAM2, ADAM, alpha4, ATP2B4 gene, a CatSper gene subunit, CatSper1, CatSper2, CatSper3, Catsper4, CatSperbeta, CatSpergamma, CatSperdelta, Clamegin, Complexin-I, Sertoli cell androgen receptor, Gasz, Ra175, Cib1, Cnot7, Zmynd15, CKs2, and Smcp.

7. The animal of claim 1 wherein the target gene is necessary for spermatogenesis, wherein disruption of the gene selectively inhibits spermatogenesis.

8. The animal of claim 7 wherein the target gene comprises Testis expressed gene 11 (Tex11).

9. The animal of claim 1 wherein the target gene is necessary for sperm motility, sperm acrosome fusion, or sperm syngamy, wherein disruption of the target gene selectively inhibits one or more of sperm motility, sperm acrosome fusion, or sperm syngamy.

10. The animal of claim 9 wherein disruption of the target gene selectively inhibits sperm motility and the gene is chosen from the group consisting of TENR, ADAM1a, ADAM3, Atp1a4, and ATP2B4.

11. The animal of claim 9 wherein disruption of the target gene selectively inhibits sperm acrosome fusion and the gene is chosen from the group consisting of ADAM2, ADAM3, CatSper, Clamegin, and Complexin-I.

12. The animal of claim 1 wherein the animal is chosen from the group consisting of non-human vertebrates, non-human primates, cattle, horse, swine, sheep, chicken, avian, rabbit, goats, dog, cat, and fish.

13. The animal of claim 1 being unable to produce functional sperm.

14. The animal of claim 13 wherein the target gene comprises DAZL.

15. The animal of claim 1 being a recipient of donor cells that give rise to functional donor sperm having a haploid donor chromosomal complement of the donor.

16. A process of preparing cells of an animal comprising:
introducing, into an organism chosen from the group consisting of a nonhuman cell and a nonhuman embryo, an agent that specifically binds to a chromosomal target site of the cell to disrupt a gene to selectively disrupt gametogenesis, with the agent being chosen from the group consisting of a targeting endonuclease, a RNA-guided nuclease, and a recombinase fusion protein.

17. The process of claim 16 wherein the agent is the targeted endonuclease and comprises a TALEN or a TALEN pair that comprises a sequence to specifically bind the chromosomal target site.

18. The process of claim 16 further comprising introducing a nucleic acid into the organism, wherein the nucleic acid sequence is introduced into the genome of the organism at the chromosomal target site.

19. The process of claim 16 wherein the cell is chosen from the group consisting of an in vitro cell, an in vitro primary cell, a zygote, an oocyte, a gametogenic cell, a sperm cell, an oocyte, a stem cell, and a zygote.

20. The process of claim 16 further comprising introducing a nucleic acid template into the cell, with the template having ends that are substantially homologous to ends produced by the break, wherein the nucleic acid template sequence is introduced into the genome of the organism at the chromosomal target site.

21. The process of claim 16 wherein the animal is chosen from the group consisting non-human vertebrates, non-human primates, cattle, horse, swine, sheep, chicken, avian, rabbit, goats, dog, cat, laboratory animal, and fish.

22. The process of claim 16 wherein the disrupted gene is chosen from the group consisting of DAZL, vasa, CatSper, KCNU1, DNAH8, and Testis expressed gene 11, TENR, ADAM1a, ADAM2, ADAM, alpha4, ATP2B4 gene, a CatSper gene subunit, CatSper1, CatSper2, CatSper3, Catsper4, CatSperbeta, CatSpergamma, CatSperdelta, Clamegin, Complexin-I, Sertoli cell androgen receptor, Gasz, Ra175, Cib1, Cnot7, Zmynd15, CKs2, and Smcp.

23. An in vitro cell comprising an agent that specifically binds to a chromosomal target site of the cell and causes a double-stranded DNA break to disrupt a gene to selectively disrupt gametogenesis, with the agent being chosen from the group consisting of a targeting endonuclease, RNA-guided nuclease, and a recombinase fusion protein.

24. A genetically modified livestock animal comprising a genomic modification to a Y chromosome, with the modification comprising an insertion, a deletion, or a substitution of one or more bases of the chromosome.

25. A genetically modified livestock animal, the animal comprising an exogenous gene on a chromosome, the gene being under control of a gene expression element that is selectively activated in gametogenesis.

26. The animal of claim 25 wherein the exogenous gene inactivates a gene selectively required for production of a male progeny, and sexual reproduction of the animal produces only female progeny.

27. The animal of claim 25 wherein the exogenous gene inactivates a gene selectively required for production of a female progeny, and sexual reproduction of the animal produces only male progeny.

28. The animal of claim 25 wherein the exogenous gene expresses a factor that is fatal to a cell to thereby destroy only male or female gametes.

29. The animal of claim 25 being a male or female that is genetically sterile, with the exogenous gene expressing a factor that interferes with a second gene that is selective for spermatogenesis or oogenesis, respectively, thereby preventing successful sexual reproduction by the animal.

30. The animal of claim 29 wherein interference with the second gene selectively inhibits sperm motility, sperm acrosome fusion, or sperm syngamy.
